SEQUENCE LISTING (9 sequences)

SEQ ID NO: 1 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 2 - protein, 136 aa, Homo sapiens [sequence omitted]

SEQ ID NO: 3 - protein, 136 aa, Homo sapiens
MARTKQTARKSTGGKAPRKQLATKVARKSAPATGGVKKPHRYRPGTVALREIRRYQKSTELLIRKLPFQRLMREIAQDFKTDLRFQSSAVMALQEACESYLVGLFEDTNLCVIHAKRVTIMPKDIQLARRIRGERA

SEQ ID NO: 4 - protein, 136 aa, Homo sapiens
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 5 - protein, 135 aa, Homo sapiens
MARTKQTARKSTGGKAPRKQLATKAARKSTPSTCGVKPHRYRPGTVALREIRRYQKSTELLIRKLPFQRLVREIAQDFNTDLRFQSAAVGALQEASEAYLVGLLEDTNLCAIHAKRVTIMPKDIQLARRIRGERA

SEQ ID NO: 6 - protein, 147 aa, Homo sapiens
MARTKQTARKATAWQAPRKPLATKAARKRASPTGGIKKPHRYKPGTLALREIRKYQKSTQLLLRKLPFQRLVREIAQAISPDLRFQSAAIGALQEASEAYLVQLFEDTNLCAIHARRVTIMPRDMQLARRLRGEGAGEPTLLGNLAL

SEQ ID NO: 7 - protein, 136 aa, Homo sapiens
MARTKQTARKATAWQAPRKPLATKAAGKRAPPTGGIKKPHRYKPGTLALREIRKYQKSTQLLLRKLPFQRLVREIAQAISPDLRFQSAAIGALQEASEAYLVQLFEDTNLCAIHARRVTIMPRDMQLARRLRREGP

SEQ ID NO: 8 - protein, 8 aa, Homo sapiens
SAPSTGGV

SEQ ID NO: 9 - protein, 8 aa, Homo sapiens
SAPATGGV

(19) United States
(12) Patent Application Publication
(10) Pub. No.: US 2026/0072040 A1
(43) Pub. Date: Mar. 12, 2026

(54) METHOD FOR MEASURING CELL FREE CHROMATIN

(71) Applicant: Belgian Volition SRL, Isnes (BE)

(72) Inventor: Jacob Vincent MICALLEF, Isnes (BE)

(21) Appl. No.: 19/106,442

(22) PCT Filed: Aug. 25, 2023
(86) PCT No.: PCT/EP2023/073350

§ 371 Date: Feb. 25, 2025

Related U.S. Application Data
(60) Provisional application No. 63/400,957, filed on Aug. 25, 2022.

(51) Int. Cl. G01N 33/68; G01N 33/574

(52) U.S. Cl. CPC G01N 33/6875; G01N 33/57585; G01N 2800/2814; G01N 2800/325

(57) ABSTRACT

The invention relates to methods and uses of cell free histone H3 isoforms H3.1, H13.2, H3t and/or H3.3 (or cell free nucleosomes containing said isoforms) of determining the origin of a cell free histone or cell free nucleosome in a body fluid sample as originating from a dividing or non-dividing cell.

### FIELD OF THE INVENTION

This invention relates to the use of cell free histone H3 isoforms H3.1, H3.2, H3t and/or H3.3 or cell free nucleosomes containing histone H3 isoforms H3.1, H3.2, H3t and/or H3.3 or cell free extracellular traps containing histone H3 isoforms H3.1, H3.2, H3t and/or H3.3, in a body fluid as a biomarker to assess the disease condition of a subject.

### BACKGROUND OF THE INVENTION

Cell free chromatin fragments, including cell free histones, cell free nucleosomes and cell free DNA (cfDNA) fragments, have been observed in many different body fluids including blood, serum, plasma, cerebrospinal fluid, sputum, faeces, urine, bronchial alveolar fluid. These cell free chromatin fragments originate predominantly by release from dead or dying cells into the extracellular space. The human body is thought to comprise some 200 different cell types and cell free chromatin fragments may, in principle, be derived from any of these.

The majority of cfDNA in the blood circulates in the form of cell free nucleosomes. The nucleosome is the basic unit of chromatin structure and consists of a protein complex of 8 highly conserved core histones (comprising of a pair of each of the histones H2A, H2B, H3, and H4). Around this complex is wrapped approximately 147 base pairs (bp) of DNA. Another histone, H1 or H5, acts as a linker and is involved in chromatin compaction. The DNA is wound around consecutive nucleosomes connected by additional linker DNA in a structure often said to resemble “beads on a string” and this forms the basic structure of open or euchromatin. Cell free di-, tri- or oligo-nucleosomes comprise a string of 2, 3 or more nucleosomes connected by a fragment of DNA including multiples of approximately 147 bp-200 bp DNA up to several thousand base pairs including linker DNA. Elevated levels of cell free nucleosomes or cfDNA in a biofluid such as blood, may indicate excessive cell death of the tissue of origin and therefore be clinically useful as an indicator of the health or disease state that tissue. However, determining the tissue of origin of a cell free chromatin fragment is problematic because nucleosomes share a common structure and cells of different tissues in the same subject have the same DNA sequence.

In addition, the most common body fluid analysed is blood and the majority of cfDNA fragments present in the circulation are of haematopoietic origin. Therefore, not only are circulating cfDNA fragments or nucleosomes originating from brain, liver, kidney, lung, heart or other tissues of interest all derived from cells with the same DNA sequence, but they are diluted into a large pool of nucleosomes containing cfDNA derived from white blood cells which also have the same DNA sequence. For these reasons, the cellular origin of a particular circulating cfDNA fragment cannot be identified on the basis of DNA sequence alone. In order to identify the cellular origin of the circulating cfDNA fragment additional and often complex further analysis such as fragmentomics is required.

In the limited cases where the tissue of origin is clear, cfDNA sequence information is clinically useful. For example, blood samples collected from pregnant subjects contain cell free cfDNA originating from both the mother and the fetus (or placenta). As the mother and fetus have different DNA sequences, the two cfDNA origins are simple to distinguish on the basis of DNA sequence. Fetal abnormalities or inherited diseases can therefore be detected non-invasively by sequencing of maternal cfDNA without biopsy of the fetus or the amniotic fluid (Guseh; 2020).

The health of donor organs transplanted into the body of a recipient may be investigated by sequencing of recipient cfDNA. Circulating cfDNA fragments with sequences corresponding to the genome sequence of the donor, in a blood sample collected from the recipient, must originate from the transplanted organ and elevated levels indicate an insult to, or deterioration of, that organ and are predictive of organ rejection (Thongprayoon et al. 2020).

Cancer is associated with DNA sequence mutations. Mutated circulating cfDNA sequences therefore likely originate from cancer cells. Analysis of cfDNA sequence in blood samples collected from patients diagnosed with cancer is used clinically as a basis for personalised treatment selection by identifying sequences predictive of the efficacy of, or resistance to, certain drugs or therapies (Polivka et al. 2016).

As all cancer cells are thought to contain DNA sequence mutations, the sequencing of circulating cfDNA in blood samples collected from patients has been investigated extensively as the basis of a blood test for the detection of cancer. Whilst cfDNA and nucleosome levels are often elevated in subjects diagnosed with late stage cancer, the detection of early stage cancer by means of identifying cancer associated mutations in plasma cfDNA has not proved successful to date and is not routinely used for clinical diagnosis (Pantel; 2016). This is because cancer cell derived cfDNA is a small proportion of total cfDNA and is, for the most part, identical in sequence to that derived from healthy cells.

Workers in the field of cfDNA in cancer have employed a variety of methods to try to overcome these limitations to determine the tissue of origin of circulating cfDNA in plasma. As most cfDNA fragments circulate in the form of nucleosomes, one method involves extensive sequencing of cfDNA to determine the nucleosome fragmentation pattern present. As global genome nucleosome positioning is different for different cell types, the nucleosome fragmentation pattern obtained can be compared to known fragmentation patterns associated with different cell types to identify the tissue of origin (Snyder et al. 2016) in a process known as fragmentomics. Another method involves sequencing for methylated cfDNA and comparing cfDNA methylation patterns obtained with known methylation patterns associated with different cell types to identify the tissue of origin (Barefoot et al. 2021). However, such methods are not suitable for routine clinical use in most hospital laboratories as they are highly technically demanding, slow and very high cost.

As cell free nucleosomes comprise a common histone-DNA nucleoprotein complex, cell free nucleosomes measured in blood, or other body fluids, may similarly originate from any tissue or tissues. Therefore, the finding of an elevated cell free nucleosome level is, as for an elevated level of cfDNA, a non-specific indicator of cell death. This limits the utility of general cell free nucleosome measurements for determining the health or disease of a tissue of origin.

A method for the identification of the tissue of origin of a cell free histone or cell free nucleosome in a body fluid is needed to enable the clinical utility of cell free histone or cell free nucleosome measurements and cfDNA analyses. We now report a method with multiple clinical applications which can distinguish between the main sources of cell free histones or cell free nucleosomes and cfDNA in a body fluid.

### SUMMARY OF THE INVENTION

According to a first aspect of the invention, there is provided a method of determining the origin of a cell free histone or cell free nucleosome in a body fluid sample as originating from a dividing or non-dividing cell comprising determining the H3 isoform composition of the cell free histone or cell free nucleosome wherein a cell free histone or cell free nucleosome comprising a H3.3 histone protein originates from a non-dividing cell and/or a cell free histone or cell free nucleosome comprising a H3.1, H3.2 or H3t histone protein originates from a dividing cell.

According to another aspect of the invention, there is provided a method of detecting a cell free histone or cell free nucleosome derived from a non-dividing cell comprising contacting a body fluid sample obtained from an individual with a binding agent which specifically binds to a H3.3 histone protein and wherein binding of said binding agent to said H3.3 histone protein is indicative that the cell free histone or cell free nucleosome is derived from a non-dividing cell.

According to a another aspect of the invention, there is provided a method of detecting whether a cell free histone or cell free nucleosome is derived from a dividing or a non-dividing cell comprising contacting a body fluid sample obtained from an individual with a binding agent which specifically binds to a H3.3 histone protein and wherein binding of said binding agent to said H3.3 histone protein is indicative that the cell free histone or cell free nucleosome is derived from a non-dividing cell.

According to another aspect of the invention, there is provided a method of detecting whether a cell free histone or cell free nucleosome is derived from a dividing or a non-dividing cell comprising contacting a body fluid sample obtained from an individual with a binding agent which specifically binds to a H3.1, H3.2 or H3t histone protein and wherein binding of said binding agent to said H3.1, H3.2 or H3t histone protein is indicative that the cell free histone or cell free nucleosome is derived from a dividing cell.

According to another aspect of the invention, there is provided a method of detecting whether a cell free histone or cell free nucleosome is derived from a dividing cell or a non-dividing cell, comprising contacting a body fluid sample obtained from an individual with a first binding agent which specifically binds to a H3.1, H3.2 or H3t histone protein and a second binding agent which specifically binds to a H3.3 histone protein, and detecting a whether a cell free histone or cell free nucleosome is derived from a non-dividing cell or a dividing cell based on the presence or level of such binding to the H3.1, H3.2 or H3t histone protein and/or to the H3.3 histone protein, wherein:
(i) the presence of binding to the H3.3 histone protein or an increased level of binding to the H3.3 histone protein relative to the level of binding to the H3.1, H3.2 or H3t histone protein is indicative that the cell free histone or cell free nucleosome is derived from a non-dividing cell; and
(ii) the presence of binding to the H3.1, H3.2 or H3t histone protein or an increased level of binding to the H3.1, H3.2 or H3t histone protein relative to the level of binding to the H3.3 histone protein is indicative that the cell free histone or cell free nucleosome is derived from a dividing cell.

According to another aspect of the invention, there is provided a method of assessing a disease condition in an individual, comprising contacting a body fluid sample obtained from the individual with a first binding agent which specifically binds to a H3.1, H3.2 or H3t histone protein and/or a second binding agent which specifically binds to a H3.3 histone protein, and assessing the disease condition based on the presence or level of such binding to the H3.1, H3.2 or H3t histone protein and/or the H3.3 histone protein, wherein:
(i) the presence of binding to the H3.1, H3.2 or H3t histone protein or an increased level of binding to the H3.1, H3.2 or H3t histone protein relative to the level of binding to the H3.3 binding protein is indicative that the disease condition is associated with inflammation;
(ii) the presence of binding to the H3.1, H3.2 or H3t histone protein or an increased level of binding to the H3.1, H3.2 or H3t histone protein relative to the level of binding to the H3.3 binding protein is indicative that the disease condition is associated with neutrophil extracellular traps (NETs) or extracellular traps (ETs); and/or
(iii) the presence of binding to the H3.3 histone protein or an increased level of binding to the H3.3 histone protein relative to the level of binding to the H3.1, H3.2 or H3t histone protein is indicative that the disease condition is characterised by death of non-dividing cells.

According to another aspect of the invention, there is provided a method of detecting, diagnosing or monitoring a disease condition in a subject, comprising contacting a body fluid sample obtained from the subject with a binding agent which binds specifically to a H3.3 histone protein to detect, diagnose or monitor the disease condition and wherein the presence of binding to the binding agent is indicative that the disease condition is characterised by death of non-dividing cells and wherein the disease condition is an infection or a reaction to an infection, such as sepsis.

According to another aspect of the invention, there is provided a method of assessing a disease condition of the central nervous system (CNS) in an individual, comprising contacting a body fluid sample obtained from the individual with a first binding agent which specifically binds to a H3.1, H3.2 or H3t histone protein and a second binding agent which specifically binds to a H3.3 histone protein, and assessing the disease condition based on the presence or level of such binding to the H3.1, H3.2 or H3t histone protein and/or the H3.3 histone protein, wherein:
(i) the presence of binding to the H3.1, H3.2 or H3t histone protein or an increased level of binding to the H3.1, H3.2 or H3t histone protein relative to the level of binding to the H3.3 binding protein is indicative that the disease condition of the brain or CNS is associated with inflammation and/or NETs; and/or
(ii) the presence of binding to the H3.3 histone protein or an increased level of binding to the H3.3 histone protein relative to the level of binding to the H3.1, H3.2 or H3t histone protein is indicative that the disease condition of the brain or CNS is characterised by death of cells of the brain or CNS.

According to another aspect of the invention, there is provided a method of detecting, diagnosing or monitoring a disease condition of the CNS in a subject, comprising contacting a body fluid sample obtained from the subject with a binding agent which binds specifically to a H3.3 histone protein to detect, diagnose or monitor the disease condition of the CNS.

According to another aspect of the invention, there is provided a method of detecting, diagnosing or monitoring a disease condition of the CNS in a subject, comprising contacting a body fluid sample obtained from the subject with a first binding agent which specifically binds to a H3.1, H3.2 or H3t histone protein and a second binding agent which specifically binds to a H3.3 histone protein, and assessing the disease condition based on the presence or level of such binding to the H3.1, H3.2 or H3t histone protein and/or the H3.3 histone protein.

According to another aspect of the invention, there is provided a method for detecting, diagnosing or monitoring a tumour of the CNS in a subject, comprising contacting a CSF sample obtained from the subject with a binding agent which specifically binds to a H3.1, H3.2 or H3t histone protein to detect, diagnose or monitor the disease condition of the CNS.

According to another aspect of the invention, there is provided a method for isolating nucleosomes and/or nucleic acid from a tumour of the CNS, comprising (i) contacting a CSF sample obtained from a subject with a binding agent which specifically binds to a H3.1, H3.2 or H3t histone protein characterised by a nucleosome; (ii) isolating the nucleosomes bound to the binding agent; and (iii) optionally extracting nucleic acid characterised by the nucleosomes isolated in step (ii).

According to another aspect of the invention, there is provided a method for isolating nucleosomes and/or nucleic acid from a tumour of the CNS, comprising (i) contacting a CSF sample obtained from a subject with a binding agent which specifically binds to a H3.3 histone protein characterised by a nucleosome; (ii) isolating the nucleosomes not bound to the binding agent; and (iii) optionally extracting the nucleic acid characterised by the nucleosomes isolated in step (ii).

According to another aspect of the invention, there is provided a method for isolating nucleosomes and/or nucleic acid from a tumour, comprising (i) contacting a body fluid sample obtained from a subject with a binding agent which specifically binds to a H3.3 histone protein characterised by a nucleosome; (ii) isolating the nucleosomes not bound to the binding agent; and (iii) optionally extracting the nucleic acid characterised by the nucleosomes isolated in step (ii).

According to another aspect of the invention, there is provided a method for the assessment of the health or suitability for transplant of a donor organ or tissue, wherein said method comprises the steps of: (i) obtaining a fluid sample from the donor organ or tissue; (ii) contacting the fluid sample with a binding agent which specifically binds to a H3.3 histone protein, and (iii) using the presence or level of such binding to the H3.3 histone protein as an indicator of the health of the donor organ or tissue or the suitability for transplant of the donor organ or tissue.

According to another aspect of the invention, there is provided a method for the assessment of the health or suitability for transplant of a donor organ or tissue, wherein said method comprises the steps of: (i) obtaining a fluid sample from the donor organ or tissue; (ii) contacting the fluid sample with a first binding agent which specifically binds to a H3.1, H3.2 or H3t histone protein and a second binding agent which specifically binds to a H3.3 histone protein, and (iii) using the presence or level of such binding to the H3.1, H3.2 or H3t histone protein and/or the H3.3 histone protein as an indicator of the health of the donor organ or tissue or the suitability for transplant of the donor organ or tissue.

According to another aspect of the invention, there is provided a method for the assessment of the health of a donor organ or tissue that has been transplanted in a subject wherein said method comprises the steps of: (i) obtaining a fluid sample from the subject; (ii) contacting the fluid sample with a binding agent which specifically binds to a H3.3 histone protein, and (iii) using the presence or level of such binding to the H3.3 histone protein as an indicator of the health of a donor organ or tissue in the subject.

According to another aspect of the invention, there is provided a method for the assessment of the health of a donor organ or tissue that has been transplanted in a subject wherein said method comprises the steps of: (i) obtaining a fluid sample from the subject; (ii) contacting the fluid sample with a first binding agent which specifically binds to a H3.1, H3.2 or H3t histone protein and a second binding agent which specifically binds to a H3.3 histone protein, and (iii) using the presence or level of such binding to the H3.1, H3.2 or H3t histone protein and/or the H3.3 histone protein as an indicator of the health of a donor organ or tissue in the subject.

According to another aspect of the invention, there is provided a method for the prediction or assessment of the risk of rejection of a donor organ or tissue that has been transplanted in a subject wherein said method comprises the steps of: (i) obtaining a fluid sample from the subject; (ii) contacting the fluid sample with a binding agent which specifically binds to a H3.3 histone protein, and (iii) using the presence or level of such binding to the H3.3 histone protein as an indicator of the risk of rejection of the donor organ or tissue in the subject.

According to another aspect of the invention, there is provided a method for the prediction or assessment of the risk of rejection of a donor organ or tissue that has been transplanted in a subject wherein said method comprises the steps of: (i) obtaining a fluid sample from the subject; (ii) contacting the fluid sample with a first binding agent which specifically binds to a H3.1, H3.2 or H3t histone protein and a second binding agent which specifically binds to a H3.3 histone protein, and (iii) using the presence or level of such binding to the H3.1, H3.2 or H3t histone protein and/or the H3.3 histone protein as an indicator of the risk of rejection of the donor organ or tissue in the subject.

According to another aspect of the invention, there is provided use of a binding agent which specifically binds to a H3.3 histone protein, to determine whether a cell free nucleosome present in a body fluid sample obtained from an individual is derived from a non-dividing cell.

According to another aspect of the invention, there is provided use of a binding agent which specifically binds to a H3.1, H3.2 or H3t histone protein, to determine whether a cell free nucleosome present in a body fluid sample obtained from an individual is derived from a dividing cell.

According to another aspect of the invention, there is provided use of a binding agent which specifically binds to a H3.3 histone protein and a binding agent which specifically binds to a H3.1, H3.2 or H3t histone protein, to determine whether a cell free nucleosome present in a body fluid sample obtained from an individual is derived from a non-dividing cell or a dividing cell, wherein:
(i) the presence of binding to the H3.3 histone protein or an increased level of binding to the H3.3 histone protein relative to the level of binding to the H3.1, H3.2 or H3t histone protein is indicative that the cell free nucleosome is derived from a non-dividing cell; and/or
(ii) the presence of binding to the H3.1, H3.2 or H3t histone protein or an increased level of binding to the H3.1, H3.2 or H3t histone protein relative to the level of binding to the H3.3 histone protein is indicative that the cell free nucleosome is derived from a dividing cell.

According to another aspect of the invention, there is provided a kit comprising a first binding agent which specifically binds to a H3.1, H3.2 or H3t histone protein and a second binding agent which specifically binds to a H3.3 histone protein, for use in assessing a disease condition in an individual.

### BRIEF DESCRIPTION OF THE FIGURES

FIG. 1: Standard curve produced by chemiluminescence labelled sandwich immunoassay for nucleosomes containing histone isoforms H3.1, H3.2 and/or H3t (referred to as “H3.1-nucleosomes”). Recombinant nucleosomes containing the histone isoform H3.3, purchased from 2 different commercial suppliers, produced no signal in the assay. A histone octamer containing histone H3.1 but no DNA (i.e., not an intact nucleosome) produced no signal in this assay. Recombinant nucleosomes containing histone isoform H3.1 (purchased from a different supplier than the calibrant supplier) produced a signal in the H3.1-nucleosome assay whether or not those nucleosomes also included a variety of histone post-translational modifications. Assay signal was also observed for wild type nucleosomes derived from Hela cells.

FIG. 2: Standard curve produced by chemiluminescence labelled sandwich immunoassay for nucleosomes containing histone isoform H3.3 (referred to as “H3.3-nucleosomes”). Commercially available recombinant mononucleosomes or polynucleosomes containing the histone isoform H3.1 produced no signal in this assay. Nucleosomes derived from HEK293 cells (immortalized human embryonic kidney cells) also produced no signal in the assay. An assay signal was produced by recombinant nucleosomes containing histone isoform H3.3 (purchased from a different supplier than the calibrant supplier).

FIG. 3: Plasma EDTA measurements for cell free nucleosomes containing histone H3 isoform H3.1 in samples taken from healthy subjects, subjects diagnosed with Acute Lymphoblastic Leukaemia (ALL), Acute Myeloid Leukaemia (AML) or Non-Hodgkin's Lymphoma (NHL).

FIG. 4: Plasma EDTA measurements for cell free nucleosomes containing histone H3 isoform H3.3 in samples taken from 7 healthy subjects and 2 subjects diagnosed with NHL.

FIG. 5: Western Blot analysis of recombinant H3.1- and H3.3-nucleosomes as well as nucleosomes derived from Hela cancer cells and a brain tissue lysate. The 4 nucleosome/histone preparations were immunoprecipitated with an anti-histone H3.1 or an anti-histone H3.3 antibody and detected using a third antibody that binds all histone H3 isoforms. Note: the histone isoform H3.3 band for brain cell lysate is less dense than the bands observed for the nucleosome preparations. This was because a constant amount of protein was analysed and, whilst the nucleosome preparations comprised predominantly histone proteins, the brain cell lysate comprised all cell proteins and proportionately less histones.

FIG. 6: H3.3-nucleosome immunoassay results for (a) healthy subjects and patients diagnosed with Acute Myocarditis, stroke or Alzheimer's Disease; (b) patients diagnosed with liver cirrhosis or non-alcoholic steatohepatitis (NASH).

FIG. 7: H3.3-nucleosome immunoassay results for patients suffering from a traumatic brain injury.

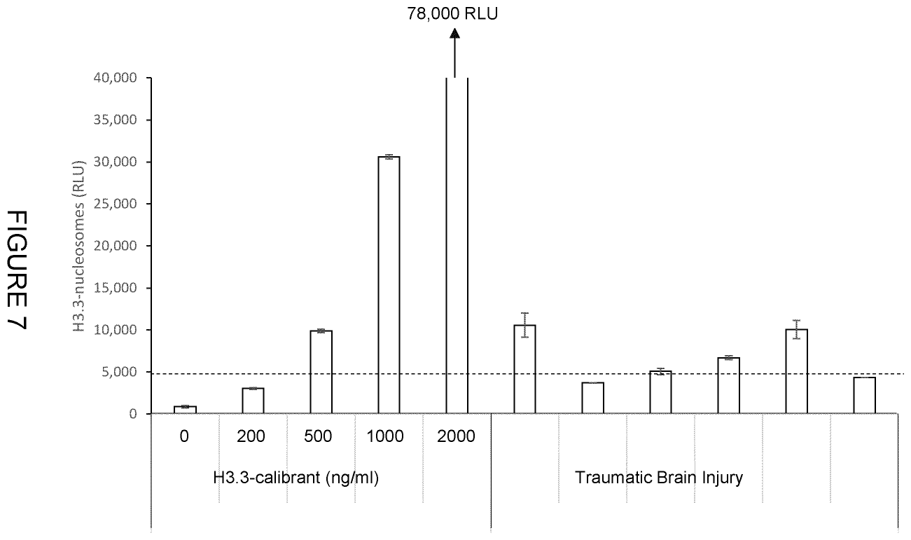

### DETAILED DESCRIPTION

The clinical utility of a biomarker often derives from the specificity of its tissue of origin. In general, blood, serum or plasma biomarkers are selected such that the marker is released into the circulation by certain diseased cells or tissues but not by healthy tissues or other diseased tissues. The presence of an elevated level of the marker in the circulation is therefore indicative of an insult to, or disease condition of, the cells of the tissue of origin. For example, circulating Prostate Specific Antigen (PSA) originates from prostate tissue and the level of PSA present in the blood is informative on prostate health. Similarly, elevated levels of liver enzymes in the circulation are informative on liver health and elevated levels of cardiac troponin-I are informative of cardiac health.

A potential advantage of cell free histones, cell free nucleosomes and cfDNA as biomarkers is that chromatin material originating from many different particular organs or tissues can be sampled and analysed by means of a single test method. This may facilitate meaningful information relating to any number of tissues simultaneously, for example through a minimally-invasive blood or other body fluid sample, without access to the organ(s) or tissue(s) themselves. However, knowledge of the tissue of origin of the cell free histone, cell free nucleosome or cfDNA present in a body fluid is required to realise the full potential of these markers. The present invention addresses this need.

Therefore according to one aspect of the present invention there is provided a method of determining the origin of a cell free nucleosome in a body fluid sample as originating from a dividing or non-dividing cell comprising determining the H3 isoform composition of the cell free nucleosome, wherein a cell free nucleosome comprising a H3.3 histone protein originates from a non-dividing cell and/or a cell free nucleosome comprising a H3.1, H3.2 or H3t histone protein originates from a dividing cell.

Therefore according to one aspect of the present invention there is provided a method of determining the origin of a cell free histone in a body fluid sample as originating from a dividing or non-dividing cell comprising determining the H3 isoform of the cell free histone, wherein a H3.3 histone protein originates from a non-dividing cell and/or a H3.1, H3.2 or H3t histone protein originates from a dividing cell.

According to one aspect of the present invention there is provided a method of detecting whether a cell free nucleosome is derived from a dividing or a non-dividing cell comprising contacting a body fluid sample obtained from an individual with a binding agent which specifically binds to a H3.3 histone protein and wherein binding of said binding agent to said H3.3 histone protein is indicative that the cell free nucleosome is derived from a non-dividing cell.

According to one aspect of the present invention there is provided a method of detecting whether a cell free histone is derived from a dividing or a non-dividing cell comprising contacting a body fluid sample obtained from an individual with a binding agent which specifically binds to a H3.3 histone protein and wherein binding of said binding agent to said H3.3 histone protein is indicative that the cell free histone is derived from a non-dividing cell.

According to another aspect of the present invention there is provided a method of detecting whether a cell free nucleosome is derived from a dividing or a non-dividing cell comprising contacting a body fluid sample obtained from an individual with a binding agent which specifically binds to a H3.1, H3.2 or H3t histone protein and wherein binding of said binding agent to said H3.1, H3.2 or H3t histone protein is indicative that the cell free nucleosome is derived from a dividing cell.

According to another aspect of the present invention there is provided a method of detecting whether a cell free histone is derived from a dividing or a non-dividing cell comprising contacting a body fluid sample obtained from an individual with a binding agent which specifically binds to a H3.1, H3.2 or H3t histone protein and wherein binding of said binding agent to said H3.1, H3.2 or H3t histone protein is indicative that the cell free histone is derived from a dividing cell.

According to another aspect of the present invention there is provided a method of detecting whether a cell free nucleosome is derived from a dividing cell or a non-dividing cell, comprising contacting a body fluid sample obtained from an individual with a first binding agent which specifically binds to a H3.1, H3.2 or H3t histone protein and a second binding agent which specifically binds to a H3.3 histone protein, and detecting a whether a cell free nucleosome is derived from a non-dividing cell or a dividing cell based on the presence or level of such binding to the H3.1, H3.2 or H3t histone protein and/or to the H3.3 histone protein, wherein:
(i) the presence of binding to the H3.3 histone protein or an increased level of binding to the H3.3 histone protein relative to the level of binding to the H3.1, H3.2 or H3t histone protein is indicative that the cell free nucleosome is derived from a non-dividing cell; and
(ii) the presence of binding to the H3.1, H3.2 or H3t histone protein or an increased level of binding to the H3.1, H3.2 or H3t histone protein relative to the level of binding to the H3.3 histone protein is indicative that the cell free nucleosome is derived from a dividing cell.

According to another aspect of the present invention there is provided a method of detecting whether a cell free histone is derived from a dividing cell or a non-dividing cell, comprising contacting a body fluid sample obtained from an individual with a first binding agent which specifically binds to a H3.1, H3.2 or H3t histone protein and a second binding agent which specifically binds to a H3.3 histone protein, and detecting a whether a cell free histone is derived from a non-dividing cell or a dividing cell based on the presence or level of such binding to the H3.1, H3.2 or H3t histone protein and/or to the H3.3 histone protein, wherein:
(i) the presence of binding to the H3.3 histone protein or an increased level of binding to the H3.3 histone protein relative to the level of binding to the H3.1, H3.2 or H3t histone protein is indicative that the cell free histone is derived from a non-dividing cell; and
(ii) the presence of binding to the H3.1, H3.2 or H3t histone protein or an increased level of binding to the H3.1, H3.2 or H3t histone protein relative to the level of binding to the H3.3 histone protein is indicative that the cell free histone is derived from a dividing cell.

In general terms a non-dividing cell is a stable or permanent cell. However, the present invention is similarly applicable to a slowly dividing cells as it is to non-dividing cells. Therefore, references herein to “non-dividing cells” includes slowly dividing cells. In general terms cells which are slowly dividing refer to cells which have a turnover of greater than or equal to 200 days. Such cells include those from adipose tissue, adrenal gland, bone marrow, heart muscle, keratinocytes, kidney, liver, lung, skeletal muscle and thyroid gland. Non-dividing cells, such as slowly dividing cells, also include cells of the central nervous system (CNS) including brain cells such as neurons and glia. Such cells of the CNS, including brain cells, are particularly those from adolescents and adults. Adolescence can be defined as the phase of life between childhood and adulthood, from ages 10 to 19.

On the other hand, a dividing cell is generally described as that which is capable of mitosis or is a labile cell. Particular examples of such cells include bone marrow, monocytes, colon, endometrium, oesophagus, keratinocytes, osteoblasts, rectum, salivary gland, smooth muscle, spleen and urinary bladder, white blood cells in particular neutrophils and other white blood cells including for example macrophages, granulocytes, mast cells, eosinophils and plasmacytoid dendritic cells.

In one embodiment the method assesses the presence of level or binding to the H3.1, H3.2 or H3t histone protein and/or the H3.3 histone protein based on the presence or level of binding to a nucleosome of which the histone protein is a component.

In one embodiment the method assesses the presence of level or binding to the H3.1, H3.2 or H3t histone protein and/or the H3.3 histone protein based on the presence or level of binding to a histone protein is a component.

In the present embodiment or other embodiments of the invention the histone protein may be detected as part of a cell nucleosome, i.e. the histone protein is a component of the cell free nucleosome.

References to “nucleosome” may refer to “cell free nucleosome” when detected in body fluid samples. It will be appreciated that the term cell free nucleosome throughout this document is intended to include any cell free chromatin fragment that includes one or more nucleosomes.

References to “histone” may refer to “cell free histone” when detected in body fluid samples. It will be appreciated that the term cell free histone throughout this document is intended to include any cell free chromatin fragment that includes one or more histones.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller; 2007). The structure of the nucleosome can vary by Post Translational Modification (PTM) of histone proteins and by the inclusion of alternative histone isoforms.

As described above, the structure of nucleosomes can vary through by the inclusion of alternative histone isoforms (also called histone variants). In more detail, the component epigenetic feature of a cell free nucleosome may be a histone isoform, such as a histone isoform of a core nucleosome, in particular a histone H3 isoform. The term “histone variant” and “histone isoform” may be used interchangeably herein. The structure of the nucleosome can also vary by the inclusion of alternative histone isoforms or variants which are different gene or splice products and have different amino acid sequences. Many histone isoforms are known in the art. Histone isoforms can be classed into a number of families which are subdivided into individual types. The nucleotide sequences of a large number of histone isoforms are known and publicly available. The nucleotide sequences of a large number of histone variants are known and publicly available for example in the National Human Genome Research Institute NHGRI Histone Database (Mariño-Ramírez et al. The Histone Database: an integrated resource for histones and histone fold-containing proteins. Database Vol. 2011. and http://genome.nhgri.nih.gov/histones/complete.shtml), the GenBank (NIH genetic sequence) Database, the EMBL Nucleotide Sequence Database and the DNA Data Bank of Japan (DDBJ). For example, isoforms of histone H2A include H2A1, H2A2, mH2A1, mH2A2, H2AX and H2AZ. In another example, histone isoforms of H3 include H3.1, H3.2, H3.3 and H3t.

PTM of histone proteins is typically an enzyme mediated chemical alteration to a histone protein in a nucleosome most commonly on the tails of core histones. Common modifications include citrullination, acetylation, methylation or ubiquitination of lysine residues as well as methylation of arginine residues and phosphorylation of serine residues. Histone modifications are highly significant changes to nucleosome structure that are known to be involved in epigenetic regulation of gene expression.

It will be appreciated that in principle the cell free nucleosome may be detected by binding to a component thereof. The term “component thereof” as used herein refers to a part of the nucleosome, i.e. the whole nucleosome does not need to be detected. However, according to the present invention we have identified that the use of a particular component of the cell free nucleosome, i.e. the claimed histone isoforms H3.3, H3.1, H3.2 and/or H3t, provides a method with multiple clinical applications and which may distinguish between sources of cell free nucleosomes and cfDNA in a body fluid.

Thus, the present invention relates particularly to use and measurement of cell free nucleosomes containing different isoforms of histone H3 as biomarkers. There are at least seven known mammalian sequence isoforms of histone H3 denoted as histone H3.1, histone H3.2, histone H3.3, histone H3.4 (H3t), histone H3.5, histone H3.X and histone H3.Y. In one embodiment, the present invention relates to the use and measurement of histone H3.3 as a biomarker. In another embodiment the present invention relates to the use of histone H3.1, H3.2 and/or H3t as a biomarker. In a further embodiment the present invention relates to the use of histone H3.3 in combination with histone H3.1, H3.2 and/or H3t.

It will be appreciated that reference to histone and histone protein are used interchangeably. Similarly, the term histone H3 and the like is used interchangeably with H3 histone.

The present invention measures cell free nucleosomes in a body fluid sample obtained from an individual. Cell free nucleosomes may be derived from dead or dying somatic cells. There are very many examples where this may occur including, without limitation, cell death during chronic illness, acute illness, infection, trauma (for example physical injury or cardiac arrest), burns or cell death due to any other insult.

Cell turnover is high in many cancers and cell free nucleosomes may be derived from dead or dying cancer cells. CfDNA derived from cancer cells is often termed circulating tumour DNA (ctDNA).

Cell free nucleosomes may be inflammatory in nature and derived from extracellular traps (ETs) produced as part of the innate immune response. ETs formation was first described for the release of neutrophil extracellular traps (NETs) by neutrophils through a process known as NETosis, but ETs are also produced by other white blood cells as well as other cells including glial cells in the brain (Wang et al. 2019). NETs are net-like structures originating from decondensed chromatin which are composed of DNA-histone complexes (i.e. nucleosomes) together with granule-derived antimicrobial peptides and enzymes such as neutrophil elastase (NE), cathepsin G and myeloperoxidase (MPO). Thus, the present invention also relates to use and measurement of cell free ETs, including NETs, containing different isoforms of histone H3 as biomarkers.

The terms “extracellular traps” or “ETs” used throughout this document are intended to include any extracellular fragment in a body fluid pertaining to an ET or ETs or a component of an ET. It will be appreciated that references to “ETs” may be used herein as a shorthand reference for neutrophil extracellular traps or other extracellular traps, as in general terms several immune cells can release chromatin and granular proteins into extracellular space in response to the stimulation, forming ETs. The cells involved in the ET formation include neutrophils, macrophages, basophils, eosinophils, microglial cells and mast cells. ETs associated with neutrophils are generally referred to as “neutrophil extracellular traps” or “NETs”. The term “NETs” used throughout this document is intended to include any extracellular fragment in a body fluid pertaining to a NET or NETs or a component of a NET.

The present invention involves identifying, detecting or measuring cell free nucleosomes containing histone isoform H3.3, or the proportion of cell free nucleosomes containing histone isoform H3.3, as originating from non-dividing cells, such as slowly dividing cells. Useful analytical methods include any measurement of nucleosomes containing histone isoform H3.3. These nucleosomes include any nucleosome also containing any other component epigenetic signals including any isoforms of histones H1, H2A, H2B, H4 or H5, any post-translational modifications, any modified nucleotides and any non-histone chromatin proteins. Various nucleosome types have been measured as described in WO2005019826, WO2013030577, WO2013030579 and WO2013084002 which are herein incorporated by reference.

Methods of the invention may also involve identifying, detecting or measuring cell free nucleosomes containing histone isoforms H3.1, H3.2 and/or H3t or the proportion of cell free nucleosomes containing histone isoform H3.1, H3.2 and/or H3t, as originating from dividing cells. In one embodiment a single measurement is able to detect or measure all three isoforms H3.1, H3.2 and H3t. In one embodiment of the invention, two measurements are made including a measure of cell free nucleosomes containing histone isoform H3.3 as well as a measure of cell free nucleosomes containing histone isoforms H3.1, H3.2 and/or H3t. This dual measurement provides information on the origin of cell free nucleosomes in a body fluid regarding the contributions of dividing and non-dividing (e.g. slowly dividing) cells to the pool of cell free nucleosomes.

Methods of the invention may also involve measuring the total level of cell free nucleosomes or nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. In one embodiment of the invention, two measurements are made including a measure of cell free nucleosomes containing histone isoform H3.3 (H3.3-nucleosomes) as well as a measure of total cell free nucleosomes or nucleosomes per se. This dual measurement provides information on the origin of cell free nucleosomes in a body fluid regarding the contributions of non-dividing (e.g. slowly dividing) cells (H3.3-nucleosomes) and dividing cells (total nucleosomes minus H3.3-nucleosomes) to the pool of cell free nucleosomes. The proportion of nucleosomes originating from non-dividing (e.g. slowly dividing) cells can be estimated as H3.3-nucleosomes/total nucleosomes. Similarly, the proportion of nucleosomes originating from dividing cells can be estimated as (H3.1-nucleosomes+H3.2-nucleosomes)/total nucleosomes. Similarly, the proportion of nucleosomes originating from non-dividing (e.g. slowly dividing) cells can be estimated as 1-(H3.1-nucleosomes+H3.2-nucleosomes)/total nucleosomes.

H3.1-nucleosomes, H3.2-nucleosomes and H3.3-nucleosomes together comprise the majority of all nucleosomes. Therefore, total cell free nucleosomes may be estimated as (H3.1-nucleosomes+H3.2-nucleosomes+H3.2-nucleosomes) and may be measured as the sum of nucleosomes detected in the two assays described herein for H3.3-nucleosomes and for (H3.1-nucleosomes+H3.2-nucleosomes+H3t-nucleosomes).

Cell free nucleosomes in a body fluid sample may conveniently be measured by immunoassay as well as by other immunochemical methods, mass spectrometry and other analytical methods. Any analytical method for the measurement of cell free nucleosomes in a body fluid may be used for the purposes of the invention.

In most cells and tissues, the majority of histone H3 present is comprised by the canonical isoforms H3.1 and H3.2. Histone isoform H3.3 is found at open chromatin sites and comprises a minor component. The remaining isoforms make up only a small proportion of the histone H3 pool.

There are two main mechanisms for the incorporation of histone H3 into nucleosomes described in the literature. The first mechanism is “replication coupled” (RC) histone incorporation. RC histone H3 incorporation occurs in dividing cells during DNA replication at the S-phase of the cell cycle to double the chromatin present and enable both daughter cells to inherit a full diploid chromosome set on mitosis.

The second mechanism is “replication independent” (RI) histone incorporation. RI histone H3 incorporation occurs continuously both in dividing cells during all phases of the cell cycle and in non-dividing or non-proliferative cells. RI histone H3 incorporation facilitates continuous turnover of histone protein molecules during the life of the cell (see for example Commerford et al. 1982, Brent and Schultz; 1999 and Ahmad and Henikoff; 2002). Ahmad and Henikoff reported in 2002 that whilst histone variant H3.3 is incorporated into chromatin throughout the cell cycle without any corresponding DNA replication and is RI, the incorporation of (canonical H3.1 and H3.2) H3 is restricted to replicating DNA and is RC.

Brain tissue comprises dividing cells in the fetus but non-dividing (e.g. very slowly dividing) cells in adult animals. In 2015, Maze et al. studied fetal and adult brain histone isoform composition in mice and humans by mass spectrometry. They reported that, the histone isoform composition of dividing brain cells in the fetus comprises predominantly canonical histone H3 isoforms H3.1 and H3.2. This is because RC incorporation of histone H3 in dividing cells preserves the histone isoform patterns present in the parent cell which, in general, comprises mostly the canonical histone H3 isoforms H3.1 and H3.2. When fetal brain cell division ceases, RC incorporation of histone H3.1 and H3.2 ceases, but RI incorporation of histone H3.3 continues. As it involves the incorporation of histone H3.3 (but not H3.1 or H3.2), RI histone incorporation results in a slow replacement of H3.1 and H3.2 proteins present in fetal brain nucleosomes by H3.3 leading to an accumulation of histone isoform H3.3 in both neuronal and glial cells in the brain.

The histone H3 present in primary mouse neurons was found to comprise <20% isoform H3.3. However, at age 24 months, histone H3.3 had accumulated to comprise 94% of the total pool of histone H3 present in the adult mouse brain and had become the dominant, if not exclusively expressed, H3 isoform.

Similar findings were reported for humans. Histone isoform H3.3 was found to constitute approximately 31% of the total H3 pool in (post mortem) fetal human brain. However, H3.3 gradually accumulated to comprise >93% of the total H3 pool in the brains of human individuals aged 14 to 72 years old.

However, the brain is not the only organ that comprises long-lived cells with long cell turnover times. Subsequent workers confirmed the findings of Maze et al. and also found that other slowly dividing somatic tissues, including liver, kidney and heart were also subject to similar age-dependent genome-wide accumulation of histone isoform H3.3. In mouse liver, kidney and brain tissue, H3.3 progressively replaced H3.1 and H3.2 isoforms during adulthood and reached saturation levels at approximately 99% of the total H3 pool, by the age of 18 months. In heart tissue the proportion of H3.3 increased to 76% of total H3 by the age of 24 months (Tvardovskiy et al. 2017).

Tvardovskiy et al. 2017 also investigated the H3.1, H3.2 and H3.3 histone isoform levels present in primary human hepatocytes derived from healthy human liver and compared them to the levels present in HepG2 hepatocellular carcinoma cells. In agreement with the findings above, they found that the histone H3 pool present in slowly dividing primary hepatocytes comprised 99% isoform H3.3. In contrast, the more rapidly dividing liver cancer cells comprised 98% isoforms H3.1 and H3.2.

As well as brain, tissues with long cell turnover times include, without limitation, adipose, adrenal, heart, kidney, liver, lung, skeletal muscle and thyroid tissue (Seim et al. 2016). Whilst not wishing to be bound by any theory we believe that cell free nucleosomes originating from these tissues will also comprise all or mostly histone H3 isoform H3.3.

Whilst not wishing to be bound by any theory we describe that cell free nucleosomes released into the circulation or other body fluid from slow dividing or non-dividing cells on cell death will comprise histone H3 isoform H3.3 (H3.3-nucleosomes). The mechanism of cell death may be any such mechanism (for example by apoptosis, necrosis or other cell death mechanism). Some cells undergo a specialised from of cell death in which they expel chromatin into the extracellular space to produce extracellular traps (ETs). When ETs in a body fluid are derived from a non-dividing (such as a slowly dividing) cell (for example glial cells), we further describe that they will be comprised all or mostly of histone H3 isoform H3.3.

A common source of nucleosomes in the circulation and many other body fluids is thought to be NETs derived from chromatin expelled by neutrophils on NETosis. In contrast to glial cells, neutrophil cells have a high cell turnover and a short life of around 7 days with a half-life in the circulation of 6-8 hours. We describe that NETs will comprise all or mostly histone H3 isoform H3.1 and H3.2.

Other white blood cells (including for example, without limitation, macrophages, granulocytes, mast cells, eosinophils or plasmacytoid dendritic cells) can also form extracellular traps (ETs). As such white cells are short lived and rapidly dividing cells, we further hypothesised that extracellular traps (ETs) expelled by white blood cells into an extracellular body fluid are also likely to comprise all or mostly histone H3 isoform H3.1 and H3.2.

Another source of circulating cell free nucleosomes is nucleosomes released from dead or dying cancer cells. As cancer cells are rapidly dividing cells, cell free nucleosomes released from cancer cells into the circulation or other body fluid are also likely to comprise all or mostly histone H3 isoform H3.1 and H3.2.

In one embodiment of the invention, we provide the use of a nucleosome assay directed to measure nucleosomes comprising histone H3 isoform H3.3. In another embodiment we provide use of a nucleosome assay directed to measure nucleosomes comprising any of histone H3 isoforms H3.1, H3.2 and/or H3t. These two assays provided herein are able to measure cell free nucleosomes that are derived from non-dividing or dividing tissue types respectively.

Therefore, in one aspect of the invention there is provided a method for measuring cell free nucleosomes comprising histone H3 isoform H3.3 in a body fluid of a human or animal subject as a measure of nucleosomes derived from a non-dividing (e.g. a slowly dividing) cell or tissue, i.e. a so-called “direct” method.

In another aspect of the invention there is provided a method for measuring cell free nucleosomes comprising histone H3 isoform H3.3 and cell free nucleosomes comprising histone H3 isoforms H3.1, H3.2 and/or H3t in a body fluid of a human or animal subject as a measure of nucleosomes derived from a non-dividing (e.g. a slowly dividing) cell or tissue or from a dividing cell or tissue respectively, i.e. a so-called “dual” and “direct” method.

In a further aspect of the invention there is provided a dual method for measuring cell free nucleosomes comprising histone H3 isoform H3.3 and total cell free nucleosomes in a body fluid of a human or animal subject. The level of nucleosomes derived from a non-dividing (e.g. a slowly dividing) cell or tissue is measured directly as the level of cell free nucleosomes comprising histone H3 isoform H3.3.

In another embodiment of the invention the level of nucleosomes derived from a dividing cell or tissue may be estimated as the difference between the total level of nucleosomes and the level of cell free nucleosomes comprising histone H3 isoform H3.3, i.e. a so-called “indirect” method.

In a further aspect of the invention there is provided a dual method for measuring cell free nucleosomes comprising histone H3 isoform H3.3 and total cell free nucleosomes in a body fluid of a human or animal subject. The proportion of nucleosomes derived from a non-dividing (e.g. a slowly dividing) cell or tissue may be estimated, for example as the [concentration of cell free nucleosomes comprising histone H3 isoform H3.3/total nucleosome concentration]. An indirect measure of the proportion of nucleosomes derived from a dividing cell or tissue may be estimated, for example as [1-concentration of cell free nucleosomes comprising histone H3 isoform H3.3/total nucleosome concentration].

In a further aspect of the invention there is provided a dual method for measuring cell free nucleosomes comprising histone H3 isoforms H3.1, H3.2 and/or H3t and total cell free nucleosomes in a body fluid of a human or animal subject. The level of nucleosomes derived from a dividing cell or tissue is measured directly as the level of cell free nucleosomes comprising histone H3 isoforms H3.1, H3.2 and/or H3t. An indirect measure of the level of nucleosomes derived from a non-dividing (e.g. a slowly dividing) cell or tissue may be estimated, for example as the difference between the total level of nucleosomes and the level of cell free nucleosomes comprising histone H3 isoforms H3.1, H3.2 and/or H3t.

In a further aspect of the invention there is provided a cell free nucleosome comprising any of histone H3 isoforms H3.1, H3.2 or H3t as a biomarker for the death of a dividing cell or the release of ETs from a dividing cell (for example NETs).

In a further aspect of the invention there is provided a cell free nucleosome comprising histone H3 isoform H3.3 as a biomarker for the death of a non-dividing cell or the release of ETs from a non-dividing cell (for example glial cell derived ETs).

It will be understood that the measurement of intact nucleosomes is not necessary for the purposes of the present invention and histone isoform levels present in a body fluid may also be measured in a free or disassociated form. Therefore, in a further aspect of the invention histone isoform H3.3 is measured in a body fluid as a measure of histones derived from non-dividing (e.g. slowly dividing) cells or tissues. Such measurements may be direct or indirect as discussed above. In another aspect histone isoforms H3.1, H3.2 and/or H3t are measured in a body fluid as a measure of histones derived from dividing cells or tissues. In a further aspect the level of total histones is measured and used in combination with the levels of histone isoform H3.3 and/or histone isoforms H3.1, H3.2 and/or H3t for indirect measurements to estimate the levels or proportions of histones derived from dividing or non-dividing (e.g. slowly dividing) cells or tissues. Methods for histone measurement are well known in the art including by isolation of histones from a body fluid sample (for example by acid extraction or by immunoprecipitation) followed by analysis of the histone isoforms present in the sample (for example by mass spectrometry, immunoassay, Western blot, other immunochemical methods or other methods). See for example Van den Ackerveken et al. 2021 and Liu et al. 2017.

### Histone Isoforms

Any method for the measurement of specific histone H3 isoforms may be used for the purposes of the present invention including mass spectrometry and immunochemical methods. Histone isoforms are distinct gene products with different amino acid sequences.

The amino acid sequences of the human histone isoforms H3.1, H3.2, H3.3 and H3t are shown below (uniport database).

H3.1:
(SEQ ID NO: 1)
MARTKQTARKSTGGKAPRKQLATKAARKSAPATGGVKKPHRYRPGTVALR
EIRRYQKSTELLIRKLPFQRLVREIAQDFKTDLRFQSSAVMALQEACEAY
LVGLFEDTNLCAIHAKRVTIMPKDIQLARRIRGERA
H3.2:
(SEQ ID NO: 2)
MARTKQTARKSTGGKAPRKQLATKAARKSAPATGGVKKPHRYRPGTVALR
EIRRYQKSTELLIRKLPFQRLVREIAQDFKTDLRFQSSAVMALQEASEAY
LVGLFEDTNLCAIHAKRVTIMPKDIQLARRIRGERA
H3t (H3.4):
(SEQ ID NO: 3)
MARTKQTARKSTGGKAPRKQLATKVARKSAPATGGVKKPHRYRPGTVALR
EIRRYQKSTELLIRKLPFQRLMREIAQDFKTDLRFQSSAVMALQEACESY
LVGLFEDTNLCVIHAKRVTIMPKDIQLARRIRGERA
H3.3:
(SEQ ID NO: 4)
MARTKQTARKSTGGKAPRKQLATKAARKSAPSTGGVKKPHRYRPGTVALR
EIRRYQKSTELLIRKLPFQRLVREIAQDFKTDLRFQSAAIGALQEASEAY
LVGLFEDTNLCAIHAKRVTIMPKDIQLARRIRGERA
H3.5 (H3.3c)
(SEQ ID NO: 5)
MARTKQTARKSTGGKAPRKQLATKAARKSTPSTCGVKPHRYRPGTVALRE
IRRYQKSTELLIRKLPFQRLVREIAQDFNTDLRFQSAAVGALQEASEAYL
VGLLEDTNLCAIHAKRVTIMPKDIQLARRIRGERA
H3.X
(SEQ ID NO: 6)
MARTKQTARKATAWQAPRKPLATKAARKRASPTGGIKKPHRYKPGTLALR
EIRKYQKSTQLLLRKLPFQRLVREIAQAISPDLRFQSAAIGALQEASEAY
LVQLFEDTNLCAIHARRVTIMPRDMQLARRLRGEGAGEPTLLGNLAL
H3.Y
(SEQ ID NO: 7)
MARTKQTARKATAWQAPRKPLATKAAGKRAPPTGGIKKPHRYKPGTLALR
EIRKYQKSTQLLLRKLPFQRLVREIAQAISPDLRFQSAAIGALQEASEAY
LVQLFEDTNLCAIHARRVTIMPRDMQLARRLRREGP

Each histone H3 isoform has a unique amino acid sequence. In principle, antibodies may be produced to target any epitope present in a H3 histone protein isoform that is absent in other isoforms to develop immunochemical assays for a specific histone isoform. However, epitope selection is more complex than this because protein structures are not linear but folded into complex shapes and only those portions of the amino acid sequence located on the outside of the folded protein structure will be exposed for potential binding. Amino acid sequences located in the interior of the folded protein structure may be inaccessible to an antibody or other binding moiety. Hence, we selected unique histone isoform epitopes which are located on the surface of the folded histone protein structure and hence available for binding.

Epitope binding availability is further complicated in the case of measuring intact nucleosomes. The histone isoform selected is one of eight histone proteins present in the combined nucleoprotein complex structure that, together with DNA, comprises the nucleosome. The unique histone isoform epitope selected, even if located on the surface of a folded histone molecule, may none-the-less be obscured or structurally affected by the presence of other histone or DNA molecules in the combined nucleoprotein complex (for example; a histone epitope may be located on the outside of an individual histone protein but in the interior of the aggregated eight histone protein complex, or covered by the DNA molecule present in the nucleosomes). Therefore, we selected unique histone isoform epitopes which are located on the surface of the folded histone protein structure and also exposed for binding in the nucleosome.

The most commonly used epitope binders in the art are antibodies or derivatives of an antibody that contain a specific binding domain. The antibody may be a polyclonal antibody or a monoclonal antibody or a fragment thereof capable of specific binding to the epitope. However, any binder capable of binding to a particular epitope may be used for the purposes of the invention. A further aspect of the invention provides binders, such as naturally occurring or chemically synthesised compounds, capable of specific binding to an epitope. A binder according to the invention may comprise an antibody or a fragment thereof, a peptide or a synthetic binder such as a plastic antibody, or an aptamer or oligonucleotide, capable of specific binding to an epitope. A binder according to the invention may be labeled with a detectable marker, such as a luminescent, fluorescent, enzyme or radioactive marker; alternatively or additionally a binder according to the invention may be labelled with an affinity tag, e.g. a biotin, avidin, streptavidin or His (e.g. hexa-His) tag. Epitope binding may also be determined using a label-free technology for example that of ForteBio Inc. The terms antibody or binder as used herein are interchangeable and refer to any moiety capable of specific binding to an epitope.

We used antibodies directed to bind to the amino acid sequence around the alanine residue (A) at position 31 in isoforms H3.1, H3.2 and H3t to bind histones and nucleosomes derived from dividing cells (underscored A in the sequences above). We used antibodies directed to bind to the amino acid sequence around the serine residue(S) at position 31 in H3.3 to bind histones and nucleosomes derived from non-dividing (e.g. slowly dividing) cells (underscored S in the sequence above).

These two epitopes were selected for a number of reasons. Firstly, the sequence SAPSTGGV (SEQ ID NO: 8) is unique to isoform H3.3 which makes it suitable for the purposes of the present invention. Secondly, the sequence SAPATGGV (SEQ ID NO: 9) is common to H3.1, H3.2 and H3t which means it will bind to all canonical H3 isoforms. Thirdly, as all or the majority of H3 in most or all tissues is comprised by H3 isoforms H3.1, H3.2 and H3.3, the combination of the two antibodies binds all or the majority of total H3 present in a body fluid sample. Fourthly, the epitope is available for binding on folded histone molecules and on intact nucleosomes. Fifthly, histones and nucleosomes containing histones, may be clipped by regulated proteolysis of the histone tail. On histone H3, clipping is reported to occur around amino acid position 21 so use of an epitope above the clip point facilitates the measurement of both clipped and un-clipped histones and nucleosomes (Yi and Kim, 2018). Sixthly, the amino acid residues in proximity to the targeted residue at position 31 (serine, alanine, proline, threonine, glycine and valine) are not commonly modified. This is important because it minimises the effect of the nearby structural variety of nucleosomes and histones on the binding of the antibodies and on the assays developed using them.

### Binding Agent

The present invention employs an agent capable of binding specifically to a histone isoform. An agent is considered to “bind specifically” to a histone isoform employed in the present invention if there is a greater than 10 fold difference, and preferably a 25, 50 or 100 fold difference between the binding of the agent to a particular histone isoform used in the present invention and another histone isoform.

The agent may be any compound capable of binding specifically to a histone isoform. The term “compound” refers to a chemical compound (naturally occurring or synthesised), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule.

A “nucleic acid” or “nucleotide sequence” is a sequence of nucleotide bases, and may be RNA, DNA or DNA-RNA hybrid sequences (including both naturally occurring and non-naturally occurring nucleotides) but is preferably either single or double stranded DNA sequences.

Preferably the agent is identifiable by screening a library of candidate compounds. Libraries of compounds may be screened in multi-well plates (e.g., 96-well plates), with a different test compound in each well. In particular, the library of candidate compounds may be a combinatorial library. A variety of combinatorial libraries of random-sequence oligonucleotides, polypeptides, or synthetic oligomers have been proposed and numbers of small-molecule libraries have also been developed. Combinatorial libraries of oligomers may be formed by a variety of solution-phase or solid-phase methods in which mixtures of different subunits are added stepwise to growing oligomers or parent compound, until a desired oligomer size is reached (typically hexapeptide or heptapeptide). A library of increasing complexity can be formed in this manner, for example, by pooling multiple choices of reagents with each additional subunit step. Alternatively, the library may be formed by solid-phase synthetic methods in which beads containing different-sequence oligomers that form the library are alternately mixed and separated, with one of a selected number of subunits being added to each group of separated beads at each step. Libraries, including combinatorial libraries are commercially available from pharmaceutical companies and specialty library suppliers.

Where the agent recognises a histone isoform used according to the present invention, the agent may comprise an MHC molecule or part thereof which comprises the peptide binding groove. Alternatively the agent may comprise an anti-peptide antibody. As used herein, “antibody” includes a whole immunoglobulin molecule or a part thereof or a bioisostere or a mimetic thereof or a derivative thereof or a combination thereof. Examples of a part thereof include: Fab, F(ab)′2, and Fv. Examples of a bioisostere include single chain Fv (ScFv) fragments, chimeric antibodies, bifunctional antibodies.

The term “mimetic” relates to any chemical which may be a peptide, polypeptide, antibody or other organic chemical which has the same binding specificity as the antibody.

The term “derivative” as used herein in relation to antibodies includes chemical modification of an antibody. Illustrative of such modifications would be replacement of hydrogen by an alkyl, acyl, or amino group.

A whole immunoglobulin molecule is divided into two regions: binding (Fab) domains that interact with the antigen and effector (Fc) domains that signal the initiation of processes such as phagocytosis. Each antibody molecule consists of two classes of polypeptide chains, light (L) chains and heavy (H) chains. A single antibody has two identical copies of the L chain and two of the H chain. The N-terminal domain from each chain forms the variable regions, which constitute the antigen-binding sites. The C-terminal domain is called the constant region. The variable domains of the H (VH) and L (VL) chains constitute an Fv unit and can interact closely to form a single chain Fv (ScFv) unit. In most H chains, a hinge region is found. This hinge region is flexible and allows the Fab binding regions to move freely relative to the rest of the molecule. The hinge region is also the place on the molecule most susceptible to the action of protease which can split the antibody into the antigen binding site (Fab) and the effector (Fc) region.

The domain structure of the antibody molecule is favourable to protein engineering, facilitating the exchange between molecules of functional domains carrying antigen-binding activities (Fabs and Fvs) or effector functions (Fc). The structure of the antibody also makes it easy to produce antibodies with an antigen recognition capacity joined to molecules such as toxins, lymphocytes or growth factors.

Chimeric antibody technology involves the transplantation of whole mouse antibody variable domains onto human antibody constant domains. Chimeric antibodies are less immunogenic than mouse antibodies but they retain their antibody specificity and show reduced HAMA responses.

In chimeric antibodies, the variable region remains completely murine. However, the structure of the antibody makes it possible to produce variable regions of comparable specificity which are predominantly human in origin. The antigen-combining site of an antibody is formed from the six complementarity-determining regions (CDRs) of the variable portions of the heavy and light chains. Each antibody domain consists of seven antiparallel [beta]-sheets forming a [beta]-barrel with loops connecting the [beta]-strands. Among the loops are the CDR regions. It is feasible to move the CDRs and their associated specificity from one scaffolding [beta]-barrel to another. This is called CDR-grafting. CDR-grafted antibodies appear in early clinical studies not to be as strongly immunogenic as either mouse or chimeric antibodies. Moreover, mutations may be made outside the CDR in order to increase the binding activity thereof, as in so-called humanised antibodies.

Fab, Fv, and single chain Fv (ScFv) fragments with VH and VL joined by a polypeptide linker exhibit specificities and affinities for antigen similar to the original monoclonal antibodies. The ScFv fusion proteins can be produced with a non-antibody molecule attached to either the amino or carboxy terminus. In these molecules, the Fv can be used for specific targeting of the attached molecule to a cell expressing the appropriate antigen. Bifunctional antibodies can also be created by engineering two different binding specificities into a single antibody chain. Bifunctional Fab, Fv and ScFv antibodies may comprise engineered domains such as CDR grafted or humanised domains.

Procedures for identifying, characterising, cloning, producing and engineering polyclonal and monoclonal antibodies and their derivatives are well established, for example using hybridomas derived from mice or transgenic mice, phage-display libraries or scFv libraries. Genes encoding immunoglobulins or immunoglobulin-like molecules can be expressed in a variety of heterologous expression systems. Large glycosylated proteins including immunoglobulins are efficiently secreted and assembled from eukaryotic cells, particularly mammalian cells. Small, non-glycosylated fragments such as Fab, Fv, or scFv fragments can be produced in functional form in mammalian cells or bacterial cells. The agent may recognise the histone isoform used in the present invention alone, or in conjunction with another compound.

The binding agent may be an aptamer or a non-immunoglobulin scaffold such as an affibody, an affilin molecule, an AdNectin, a lipocalin mutein, a DARPin, a Knottin, a Kunitz-type domain, an Avimer, a Tetranectin or a transbody.

Therefore, according to one aspect of the present invention there is provided the use of a binding agent which specifically binds to a H3.3 histone protein, to determine whether a cell free nucleosome present in a body fluid sample obtained from an individual is derived from a non-dividing cell.

According to a further aspect of the present invention, there is provided the use of a binding agent which specifically binds to a H3.1, H3.2 or H3t histone protein, to determine whether a cell free nucleosome present in a body fluid sample obtained from an individual is derived from a dividing cell.

According to a further aspect of the present invention, there is provided the use of a binding agent which specifically binds to a H3.3 histone protein and a binding agent which specifically binds to a H3.1, H3.2 or H3t histone protein, to determine whether a cell free nucleosome present in a body fluid sample obtained from an individual is derived from a non-dividing cell or a dividing cell, wherein:
(i) the presence of binding to the H3.3 histone protein or an increased level of binding to the H3.3 histone protein relative to the level of binding to the H3.1, H3.2 or H3t histone protein is indicative that the cell free nucleosome is derived from a non-dividing cell; and/or
(ii) the presence of binding to the H3.1, H3.2 or H3t histone protein or an increased level of binding to the H3.1, H3.2 or H3t histone protein relative to the level of binding to the H3.3 histone protein is indicative that the cell free nucleosome is derived from a dividing cell.

### Assays

Antibodies directed to bind to histone H3.3 or to any of histones H3.1, H3.2 or H3t may be used to measure free or disassociated histone isoforms, for example by homogeneous immunoassay, competitive immunoassay, or in a sandwich immunoassay format utilising a second antibody directed to bind to another free histone H3 epitope.

In a particular embodiment to avoid histone extraction from body fluid samples, we used antibodies as described herein to develop sandwich immunoassays for intact nucleosomes containing either histone H3.3 or any of histones H3.1, H3.2 or H3t.

To develop an assay for intact nucleosomes containing any of histones H3.1, H3.2 or H3t we coated this antibody on a solid phase and used a labelled anti-nucleosome conformational epitope antibody (which epitope is present on all or most nucleosomes) as the detection antibody. This assay can be used to measure cell free nucleosomes derived from dividing cells or tissues.

The sample may be any biological fluid (also referred to herein as body fluid) sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g., as condensed breath, or an extract or purification therefrom, or dilution thereof. In a preferred embodiment, the body fluid sample is selected from blood, serum or plasma. Biological samples also include specimens from a live subject or taken post-mortem. The samples can be prepared, for example where appropriate diluted or concentrated, and stored in the usual manner. It will be understood that methods and uses of the present invention find particular use in blood, serum or plasma samples obtained from a patient.

In one embodiment, the sample is a blood or plasma sample, in particular a plasma sample. In a further embodiment, the sample is a serum sample. In another embodiment, the body fluid sample is a CSF sample.

To develop an assay for intact nucleosomes containing histone H3.3 we coated the anti-H3.3 antibody on a solid phase and used the same labelled anti-nucleosome conformational epitope antibody as the detection antibody. This assay can be used to measure cell free nucleosomes derived from non-dividing, including slowly dividing, cells or tissues.

It is clear that measuring and/or differentiating nucleosomes or DNA of dividing or non-dividing cell origin has many applications. For illustrative purposes, some example clinical applications are discussed below. It will be understood that these non-limiting examples are not intended to be an exhaustive list of applications of methods of the invention.

### Methods of Assessing Disease Conditions

As described herein, methods of the invention can be used to measure and distinguish nucleosomes derived from non-dividing cells (including slowly dividing cells) or dividing cells in body fluid samples. This information can be used to provide better and more complete information regarding the status of the individual and lead to an improved understanding of the clinical condition, in turn leading to better clinical management.

The level of cell free nucleosomes in the circulation of healthy subjects is low. Elevated levels are reported for subjects with many different cancers of different tissues and cell types. The present invention provides an efficient and effective way of measuring and/or differentiating nucleosomes of a particular origin. Thus, the present invention can be used in the diagnosis or detection of diseases such as cancer as well as many other conditions including, without limitation, trauma, inflammatory diseases, autoimmune diseases, gastrointestinal diseases (e.g. colitis, pancreatitis, cholecystolithiasis, subileus and others), pulmonary diseases (e.g. emphysema, pneumonia and others), gynaecological diseases (e.g. ovarian cysts, endometriosis, uterus myomatosus and others), abscesses, nodular goiter, coronary heart disease and systemic lupus erythematosus (Holdenrieder et al. 2001). The present invention is particularly useful in haematological cancers, sepsis and COVID-19, as well as organ transplantation. We have, for example, previously observed particularly elevated nucleosome levels in subjects diagnosed with haematological malignancies and extremely elevated levels in subjects diagnosed with sepsis and COVID-19 where the levels on hospital admission are also predictive of organ failure and mortality (WO2021110776, WO2021186037). We have also previously observed extremely elevated levels in the perfusate of donor organs for transplant ex vivo.

According to a further aspect, there is provided a method of assessing a disease condition in an individual, comprising contacting a body fluid sample obtained from the individual with a first binding agent which specifically binds to a H3.1, H3.2 or H3t histone protein and a second binding agent which specifically binds to a H3.3 histone protein, and assessing the disease condition based on the presence or level of such binding to the H3.1, H3.2 or H3t histone protein and/or the H3.3 histone protein, wherein:
(i) the presence of binding to the H3.1, H3.2 or H3t histone protein or an increased level of binding to the H3.1, H3.2 or H3t histone protein relative to the level of binding to the H3.3 binding protein is indicative that the disease condition is associated with inflammation;
(ii) the presence of binding to the H3.1, H3.2 or H3t histone protein or an increased level of binding to the H3.1, H3.2 or H3t histone protein relative to the level of binding to the H3.3 binding protein is indicative that the disease condition is associated with neutrophil extracellular traps (NETs) or extracellular traps (ETs); and/or
(iii) the presence of binding to the H3.3 histone protein or an increased level of binding to the H3.3 histone protein relative to the level of binding to the H3.1, H3.2 or H3t histone protein is indicative that the disease condition is characterised by death of non-dividing cells.

References herein to “assessing a disease condition” refer to detecting, diagnosing or monitoring said disease condition.

References to a condition being “characterised by” a certain characteristic relates to wherein said characteristic is a distinguishing/defining feature or “hallmark” of the condition. For example, neurodegenerative diseases, such as Alzheimer's disease, Huntington's disease, and Parkinson's disease are all characterised by excessive apoptosis of neurons. Similarly, cancer is a group of diseases characterised by uncontrolled growth of abnormal cells. Whilst other diseases or conditions may be associated with said characteristic, it may not be a defining feature of the diseases or condition. For example, some cancers may be associated with inflammation, however cancer is not said to be characterised by inflammation as inflammation is not a hallmark of cancer. In one embodiment, the disease condition is associated with inflammation. In a further embodiment, the disease condition is characterised by inflammation. Inflammatory diseases include a vast array of disorders and conditions that are characterised by inflammation. Examples include allergy, asthma, autoimmune diseases, coeliac disease, glomerulonephritis, hepatitis, inflammatory bowel disease, and transplant rejection.

References herein to disease conditions characterised by death of non-dividing cells, includes disease conditions characterised by programmed and non-programmed cell death. Such diseases may also include reference to disease characterised by death of slowly dividing tissue. Cell death was historically divided into programmed cell death by apoptosis and autophagy or non-programmed cell death by necrosis. More recently, multiple cell death modalities have been described according to the stimuli, molecular mechanisms and morphologies involved including, without limitation, entosis, methuosis, paraptosis, mitoptosis, parthanatos, ferroptosis, pyroptosis, necroptosis and necrosis as well as death caused by ETs formation by non-dividing (e.g. slowly dividing) cells such as glial cells. It will be clear that cell free chromatin fragments comprising histone H3.3 released into a biological fluid as a result of the death of a non-dividing (e.g. slowly dividing) cell resulting from any cell death modality may be detected or identified by methods of the invention.

In one embodiment, the disease condition is an infection. In a further embodiment, the disease condition is sepsis. In an even further embodiment, the disease condition is COVID-19. In a further embodiment, the disease condition is associated with organ failure.

In one embodiment, the method additionally comprises determining at least one clinical parameter for the individual. This parameter can be used in the interpretation of results. Clinical parameters may include any relevant clinical information for example, without limitation, gender, weight, Body Mass Index (BMI), smoking status and dietary habits. Therefore, in one embodiment, the clinical parameter is selected from the group consisting of: family history of dementia, age, sex and body mass index (BMI).

In one embodiment, individual assay cut-off levels are used and the patient is considered positive in the panel test if individual panel assay results are above (or below if applicable) the assay cut-off level for all or a minimum number of the panel assays (for example, one of two, two of two, two of three etc). In one embodiment of the invention a decision tree model or algorithm is employed for analysis of the results.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission. Therefore, in one embodiment the method is repeated on one or more occasions and any changes in the level of H3.1, H3.2 or H3t histone protein, and/or the level of H3.3 histone protein, is used to monitor the progression of the disease condition in the individual.

### Disease Conditions of the CNS

Dementia is a syndrome associated with an ongoing decline of brain functioning including memory loss and difficulties with thinking, problem-solving or language. There are many different causes of dementia, the most common being Alzheimer's disease (AD), accounting for 50 to 75% of dementia cases. AD is a progressive neurological disorder which is thought to be caused by the abnormal build-up of proteins in and around brain cells. One of these proteins is called amyloid, deposits of which form plaques around brain cells. The other is called tau, deposits of which form tangles within brain cells.

The symptoms of AD can be split into 3 main stages: early-stage AD (mild), middle-stage AD (moderate), and late-stage AD (severe). In the early stages of AD, the main symptom is memory lapses. At this stage, someone with AD may still function independently and perform their usual day-to-day activities such as driving and working. As the disease progresses into the middle stage, memory problems will worsen and the patient usually needs support to help with everyday living. In the later stages of AD, the symptoms become increasingly severe, and may include hallucinations and delusions. Patients at this stage may need full-time care and assistance with eating, moving and personal care.

Early, accurate diagnosis is beneficial for several reasons. While there is no cure for AD, there are several medicines available to treat the symptoms. Most medicines work best for patients in the early or middle stages of the disease, therefore beginning treatment early in the disease process may help preserve daily functioning. In addition, having an early diagnosis helps patients and their families plan for the future, take care of financial and legal matters, prepare living arrangements, and develop support networks.

There is currently no simple and reliable test for diagnosing AD. Diagnosis typically involves the use of cognitive tests (e.g., Mini-Mental State Exam (MMSE)), blood tests to check for other conditions, and brain scans. Cerebrospinal fluid (CSF) biomarkers are also increasingly used to support a diagnosis of AD. CSF amyloid beta (Aβ)1-42, total tau (T-tau), and phosphorylated tau (P-tau) have utility in differentiating AD from controls and in predicting conversion from mild cognitive impairment (MCI) to AD. Consequently, these measures are included in clinical and research diagnostic criteria. However, the uptake of CSF biomarker analysis in the context of AD has been hampered by low rates of recommendation and the need to perform a lumbar puncture.

Methods for the detection, diagnosis and monitoring of other dementias and inflammatory conditions of the brain or CNS have similar limitations. These include for example diagnosis, detection and monitoring of traumatic brain injury or chronic traumatic encephalopathy (CTE) or dementia pugilistica (also known as “punch drunk”). These conditions are reported to be effects of repeated head collisions or blows or concussions occurring in contact sports such as, without limitation, boxing, martial arts, American football, soccer, rugby and others.

There is therefore a need to develop tests, preferably non-invasive, for diagnosing and monitoring inflammatory conditions of the central nervous system (CNS) such as AD or other dementia, particularly in the early stages.

Thus, according to the present invention there is provided a method of assessing a disease condition of the central nervous system (CNS) in an individual, comprising contacting a body fluid sample obtained from the individual with a first binding agent which specifically binds to a H3.1, H3.2 or H3t histone protein and a second binding agent which specifically binds to a H3.3 histone protein, and assessing the disease condition based on the presence or level of such binding to the H3.1, H3.2 or H3t histone protein and/or the H3.3 histone protein, wherein:
(i) the presence of binding to the H3.1, H3.2 or H3t histone protein or an increased level of binding to the H3.1, H3.2 or H3t histone protein relative to the level of binding to the H3.3 binding protein is indicative that the disease condition of the brain or CNS is associated with inflammation and/or NETs; and/or
(ii) the presence of binding to the H3.3 histone protein or an increased level of binding to the H3.3 histone protein relative to the level of binding to the H3.1, H3.2 or H3t histone protein is indicative that the disease condition of the brain or CNS is characterised by death of cells of the brain or CNS, including by apoptosis, necrosis, ET formation by glial cells or other cell death.

A method of detecting, diagnosing or monitoring a disease condition of the CNS in a subject, comprising contacting a body fluid sample obtained from the subject with a binding agent which binds specifically to a H3.3 histone protein to detect, diagnose or monitor the disease condition of the CNS.

A method of detecting, diagnosing or monitoring a disease condition of the CNS in a subject, comprising contacting a body fluid sample obtained from the subject with a first binding agent which specifically binds to a H3.1, H3.2 or H3t histone protein and a second binding agent which specifically binds to a H3.3 histone protein, and assessing the disease condition based on the presence or level of such binding to the H3.1, H3.2 or H3t histone protein and/or the H3.3 histone protein.

In one embodiment the disease condition of the CNS is dementia.

In another embodiment the disease condition of the CNS is AD.

In another embodiment the disease condition of the CNS is Parkinson's disease.

In another embodiment the disease condition of the CNS is traumatic brain injury or chronic traumatic encephalopathy.

### Alzheimer's Disease

In more detail, AD is a neurodegenerative disorder characterised by progressive deterioration of cognitive functions. AD is characterised by neuron and synapse loss, neuroinflammation, amyloid-beta accumulation and neurofibrillary tangles. Human and transgenic mouse model studies have shown that neutrophils adhere to blood vessels in the brain, migrate inside the brain parenchyma and release NETs inside blood vessels and in the parenchyma. The presence of NETs inside the AD brain suggests that these formations may play a key role in AD progression (Pietronigro et al. 2017). The present invention employs detection of NETs with an assay for nucleosomes containing histone H3 isoforms H3.1, H3.2 and/or H3t in the circulation or cerebrospinal fluid (CSF) of a subject may be used to detect the presence of AD and may also be used to monitor subjects diagnosed with AD (by analysis of a blood, serum or plasma sample or a CSF sample obtained from the subject respectively).

We tested plasma samples obtained from subjects diagnosed with mild, moderate or severe AD, as well as age-matched control subjects with no AD, using the assay for nucleosomes containing histone H3 isoforms H3.1, H3.2 and/or H3t and found raised levels in subjects with AD. Furthermore, the level of nucleosomes containing histone H3 isoforms H3.1, H3.2 and/or H3t was disease stage dependent. Therefore, in a further aspect of the invention there is provided a blood, serum, plasma or CSF measurement for nucleosomes containing histone isoforms H3.1, H3.2 and/or H3t to detect, or to monitor the disease progress of, AD in a subject. In one embodiment the blood, serum, plasma or CSF assay measures (free or disassociated) histone isoforms H3.1, H3.2 and/or H3t to detect, or to monitor the disease progress of, AD in a subject.

A limitation of the approach described above is that the assay for nucleosomes containing histone H3 isoforms H3.1, H3.2 and/or H3t is not specific for NETs produced by neutrophils in the brain but will detect NETs produced by neutrophils located anywhere in the body.

Although neutrophils migrate into brain tissue, the primary immune cells in the brain are the microglial cells and these cells survey the extracellular milieu for foreign antigens and play a central role in inflammation of the nervous system. Studies have shown that cerebral bacterial infection induces microglial cells, but not nerve cells, to produce ETs in the brain (Wang et al. 2019). Furthermore, microglial cells and astrocytes are reported to form structures in AD that have been termed as reactive glial nets (RGNs). Without wishing to be bound by any theory the present inventors describe that ETs formed by microglial cells would comprise all, or mostly histone H3.3. Therefore, in one aspect of the invention there is provided a method for measuring nucleosomes containing histone isoform H3.3, preferably in a blood, serum, plasma or CSF sample, to provide a test for nucleosome or ETs of brain origin to detect, or to monitor the disease progress of, AD in a subject. In one embodiment the test measures (free or disassociated) histone isoform H3.3 to provide for histones, nucleosomes or ETs of brain origin to detect, or to monitor the disease progress of, AD in a subject. An advantage provided by the present invention is that these tests will be particularly tissue specific.

In a further aspect of the invention, a 2 assay approach is provided in which both nucleosomes containing histone isoforms H3.1, H3.2 and/or H3t and nucleosomes histone isoform H3.3 are measured preferably in a blood, serum, plasma or CSF sample obtained from a subject. This will provide better and more complete information regarding the AD status of the subject with respect to glial and neutrophil derived inflammation and lead to an improved understanding of the subject's clinical condition leading to better clinical management.

### Other Diseases of the Central Nervous System

As discussed above chromatin or nucleosomes originating from dead or dying cells of the brain or central nervous system (CNS) will comprise histone isoform H3.3. A number of CNS conditions may be detected or monitored in a similar way including, without limitation, Parkinson's Disease, other dementias and Multiple Sclerosis. Therefore, in a further aspect of the invention there is provided test, preferably a blood, serum, plasma or CSF test, for nucleosomes containing histone isoform H3.3 to provide a tissue specific test for nucleosome or ETs of brain origin to detect, or to monitor the disease progress of dementia in a subject. In one embodiment the test detects or measures (free or disassociated) histone isoform H3.3 to provide a more tissue specific test for histones, nucleosomes or ETs of brain origin to detect, or to monitor the disease progress of dementia in a subject. This will provide better and more complete information regarding the status of the subject and lead to an improved understanding of the subjects clinical condition leading to better clinical management.

In a further aspect of the invention, a 2 assay approach is provided in which both nucleosomes containing histone isoforms H3.1, H3.2 and/or H3t and nucleosomes histone isoform H3.3 are preferably measured in a blood, serum, plasma or CSF sample obtained from a subject. This will provide better and more complete information regarding the status of the dementia of the subject with respect to glial and neutrophil derived inflammation and lead to an improved understanding of the subject's clinical condition leading to better clinical management.

We have also shown that plasma measurements of NETs may be used to detect other forms of dementia including those associated with brain injury such as TBI. Testing or monitoring of subjects for TBI may be useful following specific head trauma associated with accidents, for example falling or traffic accidents or concussion. Testing or monitoring of subjects at risk for CTE may be also useful on a regular basis. For example, a periodic test to monitor for cumulative brain damage or CTE among subjects playing contact sports to identify any brain damage early to enable early appropriate action or treatment, before the condition becomes symptomatic later in life.

### Tumours of the Central Nervous System (CNS)

The present invention has applicability in the detecting, diagnosing and monitoring of cancers of the CNS, including brain and intracranial tumours. In one embodiment these tumours are primary tumours.

Thus, according to one aspect of the present invention there is provided a method for detecting, diagnosing or monitoring a tumour of the CNS in a subject, comprising contacting a CSF sample obtained from the subject with a binding agent which specifically binds to a H3.1, H3.2 or H3t histone protein to detect, diagnose or monitor the disease condition of the CNS.

According to another aspect of the present invention there is provided a method for isolating nucleosomes and/or nucleic acid from a tumour of the CNS, comprising (i) contacting a CSF sample obtained from a subject with a binding agent which specifically binds to a H3.1, H3.2 or H3t histone protein associated with a nucleosome; (ii) isolating the nucleosomes bound to the binding agent; and (iii) optionally extracting nucleic acid associated with the nucleosomes isolated in step (ii).

According to another aspect of the present invention there is provided a method for isolating nucleosomes and/or nucleic acid from a tumour of the CNS, comprising (i) contacting a CSF sample obtained from a subject with a binding agent which specifically binds to a H3.3 histone protein associated with a nucleosome; (ii) isolating the nucleosomes not bound to the binding agent; and (iii) optionally extracting the nucleic acid associated with the nucleosomes isolated in step (ii).

The present invention has particular applicability for brain tumours.

As discussed above, adolescent and adult glial and neuronal brain cells comprise histone isoform H3.3. However, cancer or tumour cells are proliferative and tend to be rapidly dividing and therefore to comprise all, or predominantly histone isoforms H3.1, H3.2 and/or H3t. Therefore, according to a further aspect of the invention there is provided a method for the detection of levels, and in particular high levels, of nucleosomes containing histone H3 isoforms H3.1, H3.2 and/or H3t in the CSF using an assay of the invention to detect, or to monitor, a tumour of the brain or CNS. Furthermore, isolation of nucleosomes containing histone H3 isoforms H3.1, H3.2 and/or H3t from CSF by chromatin immunoprecipitation (ChIP) methods will yield a sample purified or enriched for nucleosomes and/or DNA of tumour origin. Therefore, according to a further aspect of the invention there is provided a ChIP method for the isolation of a purified sample of nucleosomes and/or DNA of brain/CNS tumour origin from a CSF sample obtained from a subject, comprising the steps of:
i. contacting the CSF sample or nucleosomes with an antibody or other binder of histone H3 isoforms H3.1, H3.2 and/or H3t;
ii. isolating the nucleosomes bound to the binder in step i;
iii. optionally extracting DNA from nucleosomes isolated in step ii;

In one embodiment, the enrichment of nucleosomes containing histone H3 isoforms H3.1, H3.2 and/or H3t is performed indirectly by removing nucleosomes containing histone H3 isoforms H3.3 from the sample. Unbound nucleosomes, remaining after removal of nucleosomes containing histone H3.3 by ChIP, are enriched or purified for nucleosomes of brain or CNS tumour origin. Therefore, according to a further aspect of the invention there is provided a ChIP method for the removal of nucleosomes originating from healthy brain cells (for example glial derived ETs) from a CSF sample obtained from a subject to purify, or enrich for, nucleosomes and/or DNA of brain/CNS tumour origin, comprising the steps of:
i. contacting the sample or nucleosomes with an antibody or other binder of histone H3 isoforms H3.3;
ii. isolating the nucleosomes not bound to the binder in step i;
iii. optionally extracting DNA from nucleosomes isolated in step ii;

In some embodiments the purified nucleosomes or DNA prepared in steps ii or iii above is analysed by immunoassay, sequencing or other methods.

### Other Cancers

As well as tumours of the CNS, the present invention also has applicability to tumours which are not tumours of the CNS. Such tumours include those associated with the following cancers: breast, prostate, lung, bowel, melanoma skin cancer, Non-Hodgkin's Lymphoma (NHL), kidney, head and neck, pancreas, bladder, leukaemia including Acute Lymphoblastic Leukaemia (ALL), Acute Myeloid Leukaemia (AML), uterus, oesophagus, ovary, stomach, liver, myeloma and thyroid. In one embodiment these tumours are primary tumours.

In one embodiment of the invention, there is provided a method for the enrichment of nucleosomes derived from a tumour, by removing nucleosomes containing histone H3 isoform H3.3 from a blood, serum, plasma or other body fluid sample. Unbound nucleosomes, remaining after removal of nucleosomes containing histone H3.3 by, for example, ChIP, are enriched or purified for nucleosomes of tumour origin. Therefore, according to a further aspect of the invention there is provided a ChIP method for the removal of nucleosomes originating from healthy cells (for example derived from liver, lung or kidney cells) from a sample to purify, or enrich for, nucleosomes and/or DNA of tumour origin in a plasma or other body fluid sample obtained from a subject, comprising the steps of:
i. contacting the sample or nucleosomes with an antibody or other binder of histone H3 isoform H3.3;
ii. isolating the nucleosomes not bound to the binder in step i;
iii. optionally extracting DNA from nucleosomes isolated in step ii;

In some embodiments the purified nucleosomes or DNA prepared in steps ii or iii above is analysed by immunoassay, sequencing or other methods.

### Organ Failure

According to one aspect of the present invention there is provided a method of assessing the presence or extent of organ failure in an individual, comprising contacting a body fluid sample obtained from the individual with a first binding agent which specifically binds to a H3.1, H3.2 or H3t histone protein and a second binding agent which specifically binds to a H3.3 histone protein, and assessing the disease condition based on the presence or level of such binding to the H3.1, H3.2 or H3t histone protein and/or the H3.3 histone protein, wherein:
(i) the presence of binding to the H3.1, H3.2 or H3t histone protein or an increased level of binding to the H3.1, H3.2 or H3t histone protein relative to the level of binding to the H3.3 binding protein is indicative that any organ failure is associated with inflammation;
(ii) the presence of binding to the H3.1, H3.2 or H3t histone protein or an increased level of binding to the H3.1, H3.2 or H3t histone protein relative to the level of binding to the H3.3 binding protein is indicative that any organ failure is associated with neutrophil extracellular traps (NETs) or extracellular traps (ETs); and/or
(iii) wherein the presence of binding to the H3.3 histone protein or an increased level of binding to the H3.3 histone protein relative to the level of binding to the H3.1, H3.2 or H3t histone protein is indicative that any organ failure is characterised by death of non-dividing cells.

Some tissues or organs, including liver, kidney and heart comprise slow dividing cells with sufficiently long cell lifetimes that they accumulate histone isoform H3.3. Organ failure is often characterised by cell death as well as concurrent inflammation and NETosis. Therefore, cell free nucleosomes in a body fluid sample may be derived both from the NETosis of neutrophil cells, as well as from apoptosis, necrosis or other cell death of cells of the distressed organ. It will be understood that inflammation of an organ may occur with or without significant organ cell death. Similarly, significant organ cell death may occur with little or no inflammation. Furthermore the optimal treatment for patients will vary in these various conditions. It will be understood that organ damage may occur without complete organ failure and that methods of the invention may be used to detect organ damage prior to failure. For example such use in the case of liver includes, without limitation, the detection or monitoring of non-alcoholic fatty liver disease or other liver diseases, such as hepatitis and cirrhosis. In the case of heart, use may provide information on heart tissue damage in acute or chronic cardiac conditions including heart attack. The present invention may also provide information on lupus, including systemic lupus erythematosus (SLE)—the most common form of lupus. Lupus is a long-term autoimmune disease in which the body's immune system becomes hyperactive and attacks healthy tissue. Inflammation caused by lupus can affect many different body parts and can go on to damage organs such as the kidneys, heart, lungs and the brain.

Methods of the invention can therefore be used to measure and distinguish nucleosomes derived both from NETosis as well as from organ cell death to determine the extent both of inflammation and of organ cell death. This will provide better and more complete information regarding the status of the subject and lead to an improved understanding of the subjects clinical condition leading to better clinical management.

### Infection

According to one aspect of the present invention there is provided a method of detecting, diagnosing or monitoring a disease condition in a subject, comprising contacting a body fluid sample obtained from the subject with a binding agent which binds specifically to a H3.3 histone protein to detect, diagnose or monitor the disease condition and wherein the presence of binding to the binding agent is indicative that the disease condition is characterised by death of non-dividing cells, and wherein the disease condition is an infection or a reaction to an infection such as sepsis.

According to one aspect, there is provided a method of monitoring the progress of a disease in a subject suffering from an infection or a reaction to an infection, comprising:
(i) contacting a body fluid sample obtained from the subject with a binding agent to detect or measure the level of H3.3 histone protein;
(ii) repeating step (i) on one or more occasions; and
(iii) using any changes in the level of the H3.3 histone protein to monitor the progression of the infection in the subject.

According to a further aspect, there is provided a method of assigning a risk of the development or progression of a medical complication in a subject suffering from an infection or a reaction to an infection, comprising:
(i) contacting a body fluid sample obtained from the subject with a binding agent to detect or measure the level of H3.3 histone protein; and
(ii) using the level of H3.3 histone protein detected to assign the likelihood that a medical complication will develop or progress in said subject.

According to a further aspect, there is provided a method of assigning a risk of an adverse outcome to a subject suffering from an infection or a reaction to an infection, comprising:
(i) contacting a body fluid sample obtained from the subject with a binding agent to detect or measure the level of H3.3 histone protein; and
(ii) using the level of H3.3 histone protein detected to assign the likelihood of an adverse outcome to said subject,
wherein a subject identified with a high likelihood of an adverse outcome is assigned for medical intervention.

According to a further aspect, there is provided a method of selecting a subject suffering from an infection, who is in need of medical treatment for a medical complication of the infection or a reaction to an infection, comprising:
(i) contacting a body fluid sample obtained from the subject with a binding agent to detect or measure the level of H3.3 histone protein; and
(ii) using the level of H3.3 histone protein detected to indicate the presence, progression or development of a medical complication in need of treatment in said subject.

In preferred embodiments the infection is a respiratory influenza or coronavirus infection and the medical complication is ARS, ARDS or SARS or pneumonia. Therefore in one embodiment there is provided a method of detecting a subject in need of medical treatment for pneumonia, ARS, ARDS or SARS, comprising:
(i) contacting a body fluid sample obtained from the subject with a binding agent to detect or measure the level of H3.3 histone protein; and
(ii) using the level of H3.3 histone protein as an indicator that the subject is in need of medical treatment for pneumonia, ARS, ARDS or SARS.

In other preferred embodiments the reaction to an infection is sepsis. Therefore in one embodiment there is provided a method of detecting a subject in need of medical treatment for sepsis or septic shock, comprising:
(i) contacting a body fluid sample obtained from the subject with a binding agent to detect or measure the level of H3.3 histone protein; and
(ii) using the level of H3.3 histone protein as an indicator that the subject is in need of medical treatment for sepsis or septic shock.

According to a further aspect of the invention, there is provided a method of monitoring an infection or a reaction to an infection in a subject, comprising:
(i) contacting a body fluid sample obtained from the subject with a binding agent to detect or measure the level of H3.3 histone protein;
(ii) repeating the detection or measurement of the level of H3.3 histone protein in a body fluid obtained from the subject on one or more occasions;
(iii) using any changes in the level of H3.3 histone protein to monitor the progression of the infection in the subject.

According to another aspect of the present invention there is provided a method of assessing sepsis or an infection in an individual, comprising contacting a body fluid sample obtained from the individual with a first binding agent which specifically binds to a H3.1, H3.2 or H3t histone protein and a second binding agent which specifically binds to a H3.3 histone protein, and assessing the disease condition based on the presence or level of such binding to the H3.1, H3.2 or H3t histone protein and/or the H3.3 histone protein, wherein:
(i) the presence of binding to the H3.1, H3.2 or H3t histone protein or an increased level of binding to the H3.1, H3.2 or H3t histone protein relative to the level of binding to the H3.3 binding protein is indicative that the sepsis or infection is associated with inflammation;
(ii) the presence of binding to the H3.1, H3.2 or H3t histone protein or an increased level of binding to the H3.1, H3.2 or H3t histone protein relative to the level of binding to the H3.3 binding protein is indicative that the sepsis or infection is associated with neutrophil extracellular traps (NETs) or extracellular traps (ETs); and/or
(iii) the presence of binding to the H3.3 histone protein or an increased level of binding to the H3.3 histone protein relative to the level of binding to the H3.1, H3.2 or H3t histone protein is indicative that the sepsis or infection is characterised by death of non-dividing cells.

### Infection

A primary role of NETs is to trap and kill pathogens locally and prevent spread of the infection around the body. Infection is therefore often associated with both inflammation, including NETosis, and cell death caused by the pathogen. Methods of the invention can be used to measure and distinguish nucleosomes derived both from NETosis (originating from rapidly dividing neutrophil cells) as well as from other cell death of long lived cells caused by the infection. This will provide better and more complete information regarding the status of the subject and lead to an improved understanding of the subjects' clinical condition leading to better clinical management of infections of the lung, liver, kidney, heart, CNS and other organs.

The methods of the current invention find particular use in managing infectious outbreaks. Infections can be caused by different pathogens and environmental factors. In one embodiment, the infection is a viral, bacterial, fungal or microbial infection. Bacterial infections may include mycobacterial, pneumococcal and influenzae infections, such as infections (e.g. pneumonia) caused by Streptococcus pneumoniae, Escherichia coli, Mycobacterium tuberculosis, Haemophilus influenzae and Staphylococcus aureus. In a further embodiment, the infection is a viral infection. Viral infections may include infections caused by respiratory syncytial virus (RSV), influenza type A, influenza type B and coronaviruses (e.g. COVID-19).

The infection can be defined by the tissue affected by the disease. For example, the disease may affect the heart, brain, kidneys, liver, pancreas, lungs and/or blood and the infection may be a bacterial, viral, fungal or microbial infection known to commonly affect such tissues or organs. In one embodiment, the infection is a respiratory tract infection. According to this embodiment, the infection affects the lungs, upper and/or lower respiratory tract.

Other tissues which may be affected by the disease include peripheral tissues such as limbs, hands and feet and the infection may be a bacterial infection (e.g. gangrene). In one embodiment, the infection and/or disease may affect multiple tissues or organs simultaneously. For example, the infection may be a bacterial infection of a limb, hand or foot and the disease may also affect the blood (e.g. sepsis). In one embodiment, the infection is sepsis. In another example, the disease may be cardiac or coronary failure and other tissues or organs affected by the disease may include the kidneys and renal system and/or the brain (e.g. stroke). In a yet further example, the disease may affect the lungs or the infection may be a respiratory tract infection and other tissues or organs affected may include the heart, coronary system and/or brain (e.g. heart failure, myocardial infarction and/or stroke).

### Sepsis

Sepsis is a life-threatening condition involving any or all of low blood pressure, accelerated heart rate, pain, fever with sweaty skin and feeling cold, shortness of breath and disorientation or confusion. The condition of sepsis patients may deteriorate rapidly over hours into septic shock with low blood pressure, stroke, respiratory failure, heart failure, or multiple organ failure. Without urgent treatment, sepsis may be fatal and is a major cause of death worldwide.

Immunologically, sepsis involves an extreme immune response to an infection or other insult including elevated cytokine release and elevated production and release of NETs. We have previously observed that sepsis leads to highly elevated levels of circulating cell free nucleosomes containing histone isoform H3.1 and that these levels correlate with SOFA score (sequential organ failure assessment score) in septic shock (Morimont et al. 2022). These previous findings are consistent with the hypothesis underlying the current invention that the nucleosomes were NET components that originated from a population of dividing neutrophil cells with a rapid cell turnover.

Physiologically, sepsis involves low blood pressure, poor circulation, organ failure, tissue damage and cell death. The organs most commonly affected are the lungs, kidneys, and liver. All these organs comprise slow growing cells with a low cell turnover (Seim et al. 2016). Therefore, methods of the invention can be used to measure and distinguish nucleosomes derived both from NETosis as well as from organ cell death to determine the extent of septic inflammation and of organ damage.

In one embodiment this is a dual, real-time test for the simultaneous measurement of inflammation and lung cell death in sepsis, COVID-19 infection, acute respiratory distress syndrome (ARDS), severe acute respiratory syndrome (SARS), Middle east respiratory syndrome (MERS), COVID-19 infection and similar conditions. In one embodiment the test is a point of care test. In one embodiment the test is a multiplex test.

The methods described herein also find particular use with patients suffering from systemic inflammatory response syndrome (SIRS). SIRS is an inappropriate response of the body involving an inappropriately high level of NETosis to an insult. The insult may be infectious or non-infectious including trauma, surgery, acute inflammation, ischemia, reperfusion, malignancy and many others. In one embodiment, the subject is suffering (or suspected to be suffering) from SIRS. In one embodiment, the subject is suffering (or suspected to be suffering) from an infection. In one embodiment, the subject is suffering (or suspected to be suffering) from sepsis or septic shock.

The methods of the invention will provide better and more complete information regarding the status of the subject in terms of the inappropriate inflammatory response involved as well as the organ damage caused and lead to an improved understanding of the subjects clinical condition leading to better clinical management.

### Organ Transplantation

According to one aspect of the present invention there is provided a method for the assessment of the health or suitability for transplant of a donor organ or tissue, wherein said method comprises the steps of: (i) obtaining a fluid sample from the donor organ or tissue; (ii) contacting the fluid sample with a first binding agent which specifically binds to a H3.1, H3.2 or H3t histone protein and a second binding agent which specifically binds to a H3.3 histone protein, and (iii) using the presence or level of such binding to the H3.1, H3.2 or H3t histone protein and/or the H3.3 histone protein as an indicator of the health of the donor organ or tissue or the suitability for transplant of the donor organ or tissue.

According to another aspect of the present invention there is provided a method for the assessment of the health of a donor organ or tissue that has been transplanted in a subject wherein said method comprises the steps of: (i) obtaining a fluid sample from the subject; (ii) contacting the fluid sample with a first binding agent which specifically binds to a H3.1, H3.2 or H3t histone protein and a second binding agent which specifically binds to a H3.3 histone protein, and (iii) using the presence or level of such binding to the H3.1, H3.2 or H3t histone protein and/or the H3.3 histone protein as an indicator of the health of a donor organ or tissue in the subject.

According to another aspect of the present invention there is provide a method for the prediction or assessment of the risk of rejection of a donor organ or tissue that has been transplanted in a subject wherein said method comprises the steps of: (i) obtaining a fluid sample from the subject; (ii) contacting the fluid sample with a first binding agent which specifically binds to a H3.1, H3.2 or H3t histone protein and a second binding agent which specifically binds to a H3.3 histone protein, and (iii) using the presence or level of such binding to the H3.1, H3.2 or H3t histone protein and/or the H3.3 histone protein as an indicator of the risk of rejection of the donor organ or tissue in the subject.

Once removed from the donor body an ex vivo donor organ rapidly becomes inflamed with a large concomitant release of NETs. This inflammation causes damage to the organ with loss of function and such organs are discarded as non-viable for transplantation. Removal of NETs from ex vivo lungs and livers has been shown to improve both the condition of the organ and its function. The NETs generated in the ex vivo organ can be measured as an increase in the ex vivo level of nucleosomes containing histone H3 isoform H3.1 in the perfusate of perfusion circuit including the organ. Extremely high levels of NETs have been observed in ex vivo organs (Dengu et al. 2022).

High levels of NETS are pathological and cause tissue damage and cell death. However, this damage will not be detected by measuring nucleosomes containing histone H3 isoform H3.1 as cells from liver, lung, kidney, heart are slow growing tissues that will comprise all or predominantly nucleosomes containing histone H3 isoform H3.3. Therefore, methods of the invention may be used to measure nucleosomes containing histone H3 isoform H3.3 as an indicator of tissue damage and cell death of the ex vivo organ. Dual measurement of both nucleosomes containing histone H3 isoforms H3.1 and H3.3 may be used to determine both the level of NETs present as well as the level of tissue damage caused.

Once a donor organ has been transplanted into a recipient subject in need of an organ, the recipient subject must be further monitored for the health of the transplanted organ and for organ rejection. This is currently done using slow, low throughput and very expensive cfDNA testing methods. Briefly, blood samples are taken from the recipient subject, cfDNA is then extracted from the samples and sequenced to search for any sequences that correspond to the donor DNA. As any donor DNA sequences derive from the transplanted organ, the presence of cfDNA of donor sequence is indicative of cell death of the donated organ and possible rejection.

In contrast to these cfDNA methods, the methods of the invention are low cost, high throughput, require a fraction of the blood volume needed for cfDNA sequencing and may be performed rapidly to inform patient management. Therefore, methods of the invention may be used to measure nucleosomes containing histone H3 isoform H3.3 in blood, serum, plasma or other body fluid samples obtained from the organ recipient, as an indicator of tissue damage and cell death of the transplanted organ and risk of rejection. Dual measurement of both nucleosomes containing histone H3 isoform H3.3 as well as histone H3 isoforms H3.1, H3.2 and or H3t, may be used to determine both the level of NETs formed in response to the foreign organ as well as the level of tissue damage caused to the organ. This method will be particularly suitable for use with kidney, liver, lung and heart transplantation. Therefore, in one embodiment, the donor organ is selected from a kidney, liver, heart, lung, pancreas, stomach or intestine. In one embodiment, the donor tissue is selected from whole blood, plasma, platelets, cornea, bone, tendon, skin, pancreas islets, heart valves, nerves, veins, bone marrow or stem cells. In one embodiment, the donor tissue is a limb, such as a hand, an arm or a foot.

In one embodiment, the presence of binding to the H3.3 histone protein is indicative of the presence of apoptosis or necrosis in the organ or tissue. In one embodiment, the presence of binding to the H3.1, H3.2 or H3t histone protein is indicative of the presence of inflammation. In one embodiment, the presence of binding to the H3.1, H3.2 or H3t histone protein is indicative of the presence of a condition associated with NETs or ETs.

### Methods of Treatment

We conclude here that cell free nucleosomes containing histone H3 isoforms H3.1, H3.2 and/or H3t mostly derive from neutrophils or other white blood cells or from cancer cells. The most common source is NETs or ETs reflecting inflammation.

We further conclude here that cell free nucleosomes containing histone H3 isoform H3.3 mostly derive from cell death of tissues with a long cell turnover time including CNS, liver, lung, heart and kidney reflecting organ damage or distress.

The ability to quantify both the inflammatory and tissue damage components of cell free nucleosome measurements enable specific tailored treatments for the inflammatory and tissue death involved.

The major body tissues with a long cell turnover time include the CNS, lung, liver, kidneys and heart. It will be understood that where there is an elevated level of cell death in a tissue with a long cell turnover time, it will usually be clear which tissue(s) are affected from the wider clinical picture of the patient or subject.

In one embodiment of the invention, a raised level of circulating cell free nucleosomes containing histone H3 isoform H3.3 measured in a blood, serum or plasma sample is used to indicate an elevated level of cell death in a tissue with a long cell turnover time. The tissue affected is then confirmed by a further test to measure cell free nucleosomes containing histone H3 isoform H3.3 in a different body fluid where the nature of the body fluid identifies the tissue affected. A high circulatory level may be further identified as lung in origin by confirmation of an elevated level in a sputum sample or in a bronchial alveolar lavage (BAL) fluid sample. A CNS origin may be identified by an elevated level in a CSF sample. A kidney origin may be identified by an elevated level in a urine sample.

The tissue(s) affected can also be determined or confirmed using other tests. Suitable blood tests include liver enzyme tests for liver tissue damage (e.g. alanine transferase, aspartate transferase or gamma-glutamyl transpeptidase), troponin testing for heart tissue damage and creatinine testing for kidney tissue damage. Organ function tests may also be used such as lung function tests, glomerular filtration rate determination for kidney function, cognitive function for CNS and others.

In a further aspect of the invention there is provided a method of treatment for a subject suffering, or suspected of suffering, a disease condition such as those described above, comprising the steps of:
i. obtaining a body fluid sample from the subject
ii. detecting or measuring the level of nucleosomes containing histone H3 isoform H3.3 present in the sample;
iii. using the presence or level of nucleosomes containing histone H3 isoform H3.3 present in the sample as an indicator of cell death of non-dividing (e.g. slowly dividing) tissue;
iv. optionally performing further tissue function testing; and
v. providing a treatment to alleviate the cell death of the tissue.

In a further aspect of the invention there is provided a method of treatment for a subject suffering, or suspected of suffering, a disease condition such as those described above, comprising the steps of:
i. obtaining a body fluid sample from the subject
ii. detecting or measuring the level of nucleosomes containing histone H3 isoform H3.3 present in the sample and detecting or measuring the level of nucleosomes containing any of histone H3 isoforms H3.1, H3.2 and/or H3t present in the sample
iii. using the presence or level of nucleosomes containing histone H3 isoform H3.3 present in the sample as an indicator of cell death of non-dividing (e.g. slowly dividing), tissue;
iv. using the presence or level of nucleosomes containing any of histone H3 isoforms H3.1, H3.2 and/or H3t present in the sample as an indicator of cell death of dividing tissue;
v. optionally performing further tissue function testing; and
vi. providing a treatment to alleviate the cell death of the tissue.

### Other Biomarkers

The level of cell free nucleosomes may be detected or measured as one of a panel of measurements. The panel may comprise different epigenetic features of the nucleosome as described hereinbefore (e.g., a histone isoform and a PTM). Biomarkers useful in a panel test for the detection of AD include, without limitation, amyloid-β (Aβ42), total tau (T-tau), and phosphorylated tau (P-tau).

Further, additional markers of NETs may be employed. Several proteins occur in NETs that are adducted directly or indirectly to nucleosomes. These proteins include, without limitation, myeloperoxidase (MPO), neutrophil elastase (NE), lactotransferrin, azurocidin, cathepsin G, leukocyte proteinase 3, lysozyme C, neutrophil defensin 1, neutrophil defensin 3, myeloid cell nuclear differentiation antigen, S100 calcium-binding protein A8, S100 calcium-binding protein A9, S100 calcium-binding protein A12, actin β, actin γ, alpha-actin, plastin-2, cytokeratin-10, catalase, alpha-enolase and transketolase (Urban et al., PLoS Pathogens. (2009) 10: e1000639). Any nucleosome-protein adduct that occurs in NETs is a useful adduct for the detection of elevated levels of NETs in methods of the invention. C-reactive protein (CRP) may also be adducted to nucleosomes in NETs and nucleosome-CRP adduct is therefore a useful adduct in methods of the invention.

In one embodiment, the component histone PTM is selected from citrullination or ribosylation, in particular citrullination. In a further embodiment, the histone PTM is H3 citrulline (H3cit) or H4 citrulline (H4cit). In a yet further embodiment, the histone PTM is H3cit. Therefore in a further aspect of the invention there is provided a method for the measurement of a nucleosome containing histone isoform H3.3 modified by PTM as a nucleosome derived from a non-dividing (e.g. slowly dividing) cell. For example a nucleosome containing H3.3cit or any other PTM. Similarly, there is provided a method for the measurement of a nucleosome containing histone isoform H3.1, H3.2 and/or H3t modified by PTM as a nucleosome derived from a dividing cell. For example a nucleosome containing H3.1cit, H3.2cit, H3tcit or any other PTM.

The panel of measurements may be determined from the same sample, or from different samples. In one embodiment, both markers are obtained from a blood sample. In an alternative embodiment, the first marker is obtained from a blood sample and the second marker is obtained from a different body fluid sample, such as a CSF sample.

### Measurement Methods

The present invention involves identifying, detecting or measuring cell free nucleosomes containing histone isoform H3.3, or the proportion of cell free nucleosomes containing histone isoform H3.3, as originating from non-dividing (e.g. slowly dividing) cells. Useful analytical methods include any measurement of nucleosomes containing histone isoform H3.3. These nucleosomes include any nucleosome also containing any other component epigenetic signals including any isoforms of histones H1, H2A, H2B, H4 or H5, any post-translational modifications, any modified nucleotides and any non-histone chromatin proteins. Various nucleosome types have been measured (as referenced in WO2005019826, WO2013030577, WO2013030579 and WO2013084002 which are herein incorporated by reference).

Methods of the invention may also involve identifying, detecting or measuring cell free nucleosomes containing histone isoforms H3.1, H3.2 and/or H3t or the proportion of cell free nucleosomes containing histone isoform H3.1, H3.2 and/or H3t, as originating from dividing cells. In one embodiment a single measurement is able to detect or measure all three isoforms H3.1, H3.2 and H3t. In one embodiment of the invention, two measurements are made including a measure of cell free nucleosomes containing histone isoform H3.3 as well as a measure of cell free nucleosomes containing histone isoforms H3.1, H3.2 and/or H3t. This dual measurement provides information on the origin of cell free nucleosomes in a body fluid regarding the contributions of dividing and non-dividing (e.g. slowly dividing) cells to the pool of cell free nucleosomes. In one embodiment, both markers are obtained from the same type of body fluid sample, e.g. a blood sample. In an alternative embodiment, the first marker is obtained from a blood sample and the second marker is obtained from a different body fluid sample, such as a CSF sample.

Methods of the invention may also involve measuring the total level of (cell free) nucleosomes or nucleosomes per se. References to “nucleosomes per se” refers to the total nucleosome level or concentration present in the sample, regardless of any epigenetic features the nucleosomes may or may not include. Detection of the total nucleosome level typically involves detecting a histone protein common to all nucleosomes, such as histone H4. Therefore, nucleosomes per se may be measured by detecting a core histone protein, such as histone H4. In one embodiment of the invention, two measurements are made including a measure of cell free nucleosomes containing histone isoform H3.3 (H3.3-nucleosomes) as well as a measure of total cell free nucleosomes or nucleosomes per se. This dual measurement provides information on the origin of cell free nucleosomes in a body fluid regarding the contributions of non-dividing or slowly dividing cells (H3.3-nucleosomes) and dividing cells (total nucleosomes minus H3.3-nucleosomes) to the pool of cell free nucleosomes. The proportion of nucleosomes originating from non-dividing (e.g. slowly dividing) cells can be estimated as H3.3-nucleosomes/total nucleosomes. Similarly, the proportion of nucleosomes originating from dividing cells can be estimated as (H3.1-nucleosomes+H3.2-nucleosomes)/total nucleosomes. Similarly, the proportion of nucleosomes originating from non-dividing or slowly dividing cells can be estimated as 1-(H3.1-nucleosomes+H3.2-nucleosomes)/total nucleosomes.

H3.1-nucleosomes, H3.2-nucleosomes and H3.3-nucleosomes together comprise the majority of all nucleosomes. We therefore reason that total cell free nucleosomes may be estimated as (H3.1-nucleosomes+H3.2-nucleosomes+H3.2-nucleosomes) and may be measured as the sum of nucleosomes detected in the two assays described herein for H3.3-nucleosomes and for (H3.1-nucleosomes+H3.2-nucleosomes+H3t-nucleosomes).

Cell free nucleosomes in a body fluid sample may conveniently be measured by immunoassay as well as by other immunochemical methods, mass spectrometry and other analytical methods. Any analytical method for the measurement of cell free nucleosomes in a body fluid may be used for the purposes of the invention.

In one embodiment, the level of the one or more histone biomarkers detected is compared to a control or a reference level. It will be clear to those skilled in the art that the control or reference level may be selected on a variety of basis which may include, for example, the control or reference level may be one of that associated with subjects known to be free of the disease or may be subjects with a different disease (for example, for the investigation of differential diagnosis). The “control” may comprise a healthy subject, a non-diseased subject or a subject or may be a level determined for an individual earlier in the course of diagnosis. Regarding the latter, the reference level is useful to determine changes in biomarker levels which are indicative of disease progression. Comparison with a control is well known in the field of diagnostics.

It will be understood that it is not necessary to measure healthy/non-diseased controls for comparative purposes on every occasion because once the ‘normal range’ is established it can be used as a benchmark for all subsequent tests. A normal range can be established by obtaining samples from multiple control subjects without the relevant disorder, and testing for the level of biomarker. Results (i.e. biomarker levels) for subjects suspected to have an disorder can then be examined to see if they fall within, or outside of, the respective normal range. Use of a ‘normal range’ is standard practice for the detection of disease.

In one embodiment, the level of the histone biomarker is elevated compared to a control or reference level. In methods in which more than one biomarkers are determined these levels may be compared to a reference wherein the levels or ratios of the plurality of biomarkers in comparison to the reference levels or ratios is indicative that the subject will develop the disorder or that the disorder will progress.

In other words the level of the histone biomarker is compared to a reference level. By reference level is meant a level of biomarker concentration in a sample against which a test sample is compared.

If a subject is determined to not have a disease or is at a particular stage of disease, then the invention may still be used for the purposes of monitoring any disease progression. For example, if the use comprises a blood, serum or plasma sample from a subject determined not to have dementia, then the biomarker level measurements can be repeated at another time point to establish if the biomarker level has changed.

By stage of disease is meant the severity of the disease classified largely as one of mild (early stage), moderate (mid-stage), or severe (late stage). A determination of stage of disease is important in planning treatment or proposing treatment options.

In one embodiment, there is provided a method for determining the risk of developing AD. The status of a subject may be low, medium or high risk based of calculated levels of biomarkers. The amounts of the individual biomarkers or patterns of biomarker levels are characteristic of risk state (e.g. low, medium or high). The risk of developing AD is determined by measuring relevant biomarker levels in a subject and comparing the levels with a reference level or pattern of biomarker levels associated with a specific level of risk.

In one embodiment there is provided a method for determining the stage of AD. Each stage is associated with a characteristic amounts of a biomarker (a pattern). The stage of the disease is determined by measuring the levels of the biomarkers and comparing them with reference amounts associated with a particular stage of the disease. For example, biomarker levels can be used to classify between early stage AD and non-AD, or early stage AD and late stage AD. “mild”, “moderate” and “severe” degrees of disease are indicative of increasing level of disease.

In one embodiment, there is provided a method for determining the course of disease in a subject. Disease course refers to changes in disease status over time, whether progressive (worsening) or regressive (improving). Over time, levels in biomarkers may change.

In one embodiment, there is provided a method of monitoring the progression of AD by monitoring the levels of the biomarker over time. For example, the level in a body fluid sample can be collected at a first time (T1) and compared to its level in body fluid sample collected at a second time (T2), in order to determine, quantify or predict the progression of a disorder.

References to “subject”, “individual” or “patient” are used interchangeably herein. The use, panels and methods described herein are preferably performed in vitro. References to acts carried out on a body fluid sample “obtained” from a subject are intended to encompass acts carried only a body fluid sample already obtained of “obtainable” from a subject and vice versa.

The subject may be any subject for which the methods of the present invention are desired. In one embodiment, the patient is a human patient. In one embodiment, the patient is a (non-human) animal. In some embodiments the invention encompasses uses in animals (wild or domesticated). In some embodiments, the invention relates to veterinary uses including for livestock and companion animals such as cats, dogs, horses, sheep, goats, pigs, deer, llamas, cows and cattle. It also includes an individual animals in all stages of development, e.g. it includes pre-natal testing.

In certain embodiments, the subject may be one that has been diagnosed with or is suspected of having a disease or disorder. In some embodiments, the subject may be one that is at risk for developing a disease or disorder, e.g., due to genetics, family history, exposure to toxins, etc.

In one embodiment, detection or measurement of one or more of said biomarkers comprises an immunoassay, immunochemical, mass spectroscopy, chromatographic, chromatin immunoprecipitation or biosensor method. In one embodiment, the method is performed using immunoassay. In another embodiment, the method is performed using chromatin immunoprecipitation.

In one embodiment, the method of detection or measurement comprises contacting the body fluid sample with a solid phase comprising a binding agent that detects cell free nucleosomes or a component thereof, and detecting binding to said binding agent.

In one embodiment, the detection or measurement comprises an immunoassay. In one embodiment of the invention there is provided a 2-site immunoassay method for nucleosome moieties. In particular, such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. In another embodiment of the invention, there is provided a 2-site immunoassay employing a labelled anti-nucleosome detection binding agent in combination with an immobilized anti-histone modification or anti-histone variant or anti-DNA modification or anti-adducted protein binding agent.

In one embodiment, the method of detection or measurement comprises: (i) contacting the sample with a first binding agent which binds to an epigenetic feature of a cell free nucleosome; (ii) contacting the sample bound by the first binding agent in step (i) with a second binding agent which binds to a H3.1, H3.2 or H3t; and (iii) detecting or quantifying the binding of the second binding agent in the sample. In an alternative embodiment, the method of detection or measurement comprises: (i) contacting the sample with a first binding agent which binds to H3.1, H3.2 or H3t; (ii) contacting the sample bound by the first binding agent in step (i) with a second binding agent which binds an epigenetic feature of a cell free nucleosome; and (iii) detecting or quantifying the binding of the second binding agent in the sample.

In another embodiment, the method of detection or measurement comprises: (i) contacting the sample with a first binding agent which binds to an epigenetic feature of a cell free nucleosome; (ii) contacting the sample bound by the first binding agent in step (i) with a second binding agent which binds to a H3.3; and (iii) detecting or quantifying the binding of the second binding agent in the sample. In an alternative embodiment, the method of detection or measurement comprises: (i) contacting the sample with a first binding agent which binds to H3.3; (ii) contacting the sample bound by the first binding agent in step (i) with a second binding agent which binds an epigenetic feature of a cell free nucleosome; and (iii) detecting or quantifying the binding of the second binding agent in the sample.

It will be understood that any first and second binding agents may be added to the sample separately in any order or may be added simultaneously. Furthermore, the method may be repeated on one or more occasions and any changes in the level of binding to the first binding agent and/or the second binding agent is used to monitor the progression of the disease condition in the individual

Detecting or measuring the level of the biomarker(s) may be performed using one or more reagents, such as a suitable binding agent. In one embodiment, the one or more binding agents comprises a ligand or binder specific for the desired biomarker, e.g. H3.1 and/or H3.3, or a structural/shape mimic of the biomarker or component part thereof. References to a “biomarker” as used herein may include any single biomarker moiety or a combination of individual biomarker moieties in a biomarker panel.

It will be clear to those skilled in the art that the terms “antibody”, “binder” or “ligand” in regard to any aspect of the invention is not limiting but intended to include any binder capable of binding to particular molecules or entities and that any suitable binder can be used in the method of the invention.

Methods of detecting biomarkers are known in the art. In one embodiment, the reagents comprise one or more ligands or binders. In one embodiment, the ligands or binders of the invention include naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

According to a further aspect of the invention, there is provided a kit comprising a first binding agent which specifically binds to a H3.1, H3.2 or H3t histone protein and a second binding agent which specifically binds to a H3.3 histone protein, for use in assessing a disease condition in an individual. According to a further aspect of the invention, there is provided the use of a kit for assessing a disease condition in an individual (as described herein), wherein said kit comprises a first binding agent which specifically binds to a H3.1, H3.2 or H3t histone protein and a second binding agent which specifically binds to a H3.3 histone protein. Optionally, said kit may comprise instructions for use.

A further aspect of the invention is a kit for detecting the presence of a disease state, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

In one embodiment in which the test is used to derive the progression of the severity of a disorder, the reference substance may be a body fluid sample obtained from the patient at an earlier time. In an additional embodiment, the results of this biomarker testing can be used to prescribe medication. The relative and absolute values of the biomarkers disclosed herein can be used to create a disease profile for an individual, and medication can be selected based upon this profile. Profiling results will allow further monitoring of disease progression and treatment efficacy. In addition the results will permit stratification of patients for clinical drug studies. The subject can then be given medications, which have been shown to provide therapeutic benefit for patients with similar profiles.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers of the invention.

Biomarkers for detecting the presence of a disease are essential targets for discovery of novel targets and drug molecules that retard or halt progression of the disorder. As the result for a biomarker or biomarker panel is indicative of disorder and of drug response, the biomarker is useful for identification of novel therapeutic compounds in in vitro and/or in vivo assays. Biomarkers and biomarker panels of the invention can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The term “biomarker” means a distinctive biological or biologically derived indicator of a process, event, or condition. Biomarkers can be used in methods of detection, diagnosis, e.g. clinical screening, and prognosis assessment and in monitoring the results of therapy, identifying subjects most likely to respond to a particular therapeutic treatment, drug screening and development. Biomarkers and uses thereof are valuable for identification of new drug treatments and for discovery of new targets for drug treatment.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Identifying and/or quantifying can be performed by any method suitable to identify the presence and/or amount of a specific protein in a biological sample from a subject or a purification or extract of a biological sample or a dilution thereof. In methods of the invention, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed using an immunological method, such as an immunoassay. Immunoassays include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels.

In another example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving identification and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. Detection of a biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid detection and diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

It will be understood that the embodiments described herein may be applied to all aspects of the invention, i.e. the embodiment described for the uses may equally apply to the claimed methods and so forth.

The method will now be illustrated by the following examples.

### Example 1

We developed a chemiluminescent immunoassay for nucleosomes containing histone H3 isoforms H3.1, H3.2 and/or H3t (H3.1/2/t-nucleosomes). The immunoassay used magnetic beads coated with an anti-histone H3.1/2/t-nucleosome antibody directed to bind to the epitope located around the alanine residue at position 31 of H3 isoforms H3.1, H3.2 and H3t. The labelled antibody used was an acridinium ester labelled antibody directed to bind to a nucleosome conformational epitope present in intact nucleosomes.

The immunoassay was performed using an automated immunoassay system. Briefly, calibrant or sample (50 μl) was incubated with an acridinium ester labelled anti-nucleosome antibody (50 μl) and assay buffer (100 μl) for 1800 seconds at 37° C. Magnetic beads coated with an anti-histone H3.1/2/t-nucleosomes antibody (20 μl) were added and the mixture was incubated for a further 900 seconds. The magnetic beads were then isolated, washed 3 times and magnetic bound acridinium ester was determined by luminescence output over 7000 milliseconds in RLU.

We used a solid phase capture antibody directed to bind the H3 amino acid sequence surrounding amino acid 31 of histone isoforms H3.1, H3.2 and H3t (SAPATGGV). As described above, this approach has many advantages including that the H3 amino acid at position 31 is the same in histone isoforms H3.1, H3.2 and H3t but is different in isoform H3.3, is available for binding on both an intact histone and an intact nucleosome, is located above the major H3 clipping position and is therefore included in both clipped and unclipped nucleosomes, and the adjacent amino acids are not commonly subject to PTM, thereby minimising the effect of PTM makeup on antibody binding.

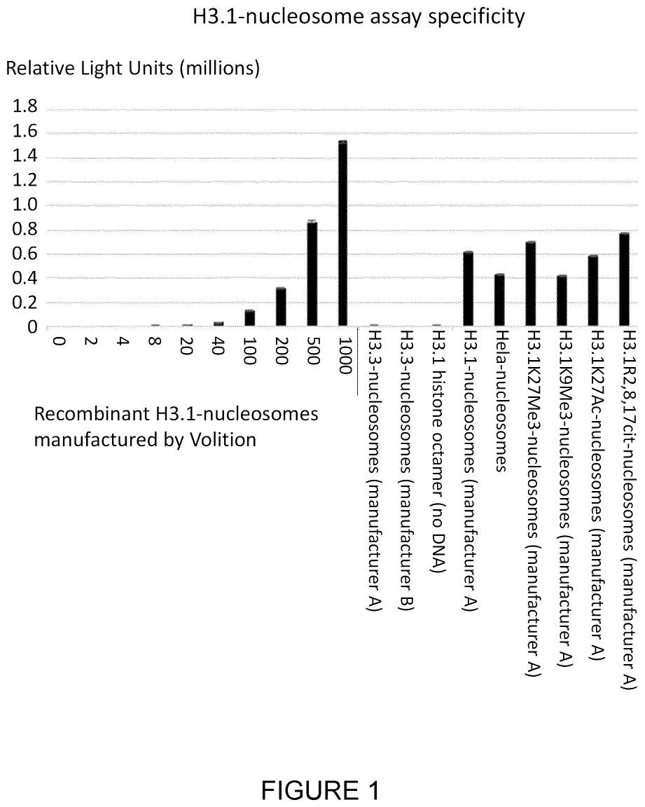

We tested the assay for specificity for the measurement of H3.1/2/t-nucleosomes whilst not detecting nucleosomes containing histone H3 isoform H3.3 (H3.3-nucleosomes). To do this we produced a standard curve using recombinant nucleosomes containing histone isoform H3.1 and tested two preparations of recombinant nucleosomes containing histone isoform H3.3, purchased from two different commercial suppliers (A and B). The preparations were diluted from the manufacturer's stated concentration to 500 ng/ml for testing. The measured results for 500 ng/ml H3.3-nucleosomes in the H3.1/2/t-nucleosome assay were 6 ng/ml and 2 ng/ml giving cross-reactivities of 1.2% and 0.4% respectively. The results are shown in FIG. 1.

To confirm that a positive signal was obtained only for intact nucleosomes and not for histones alone, including histone H3.1 even if assembled in a histone core octamer complex, we tested a preparation of H3.1-histone octamer and no significant signal (cross-reactivity 1.8%) was obtained in the H3.1/2/t-nucleosome assay (FIG. 1).

As positive controls we tested recombinant nucleosomes containing histone isoform H3.1 supplied from another manufacturer (A) and biologically derived cell free nucleosomes obtained by the digestion of chromatin from cultured Hela cancer cells. These cells are rapidly dividing and would therefore be expected to contain all or predominantly H3.1/2/t-nucleosomes and a low proportion of H3.3-nucleosomes. Both of these positive controls gave significant signals in the H3.1/2/t-nucleosome assay (FIG. 1).

We also tested commercially available recombinant H3.1-nucleosomes containing a variety of histone PTMs including H3.1K27Me3, H3.1K9Me3, H3.1K27Ac and H3.1R2,8,17citrulline. All of these modified H3.1-nucleosomes gave significant signals in the H3.1/2/t-nucleosome assay, showing that the H3.1/2/t-nucleosome assay detects a wide variety of modified or unmodified H3.1-nucleosomes (FIG. 1).

We conclude that the H3.1/2/t-nucleosome assay designed to detect and measure intact nucleosomes containing histone H3 isoforms H3.1, H3.2 and/or H3t detects these nucleosomes with little or no detection of nucleosomes containing histone H3 isoform H3.3.

We also conclude that cell free nucleosomes derived from rapidly dividing Hela cells comprise nucleosomes containing histone H3 isoforms H3.1, H3.2 and/or H3t.

### Example 2

We developed an automated immunoassay for nucleosomes containing histone H3 isoform H3.3 (H3.3-nucleosomes). We did this by using a solid phase capture antibody directed to bind the H3 amino acid sequence surrounding the serine residue located at amino acid 31 of histone H3.3 (SAPSTGGV). This approach has similar advantages to those described above in EXAMPLE 1, including that: the H3 amino acid at position 31 is the same in histone isoforms H3.1, H3.2 and H3t (alanine) but is different in isoform H3.3 (serine); it is available for binding on both an intact histone and an intact nucleosome; it is located above the major H3 clipping position and is therefore included in both clipped and unclipped nucleosomes; and the adjacent amino acids are not commonly subject to PTM, thereby minimising the effect of PTM makeup on antibody binding. We used the same labelled antibody directed to bind to a nucleosome conformational epitope present in intact nucleosomes as used in EXAMPLE 1.

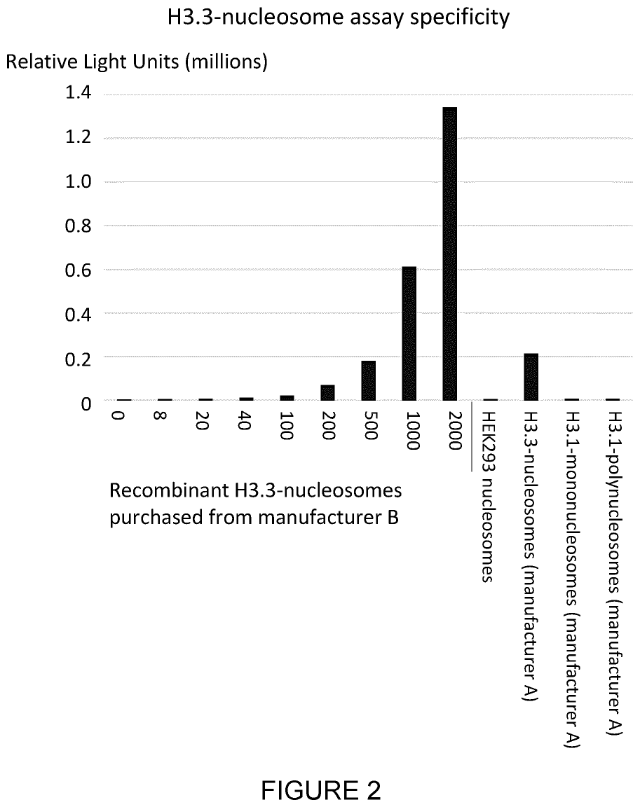

We tested the assay for specificity for the measurement of H3.3-nucleosomes whilst not detecting nucleosomes containing histone H3 isoforms H3.1, H3.2 and/or H3t (H3.1/2/t-nucleosomes). To do this we produced a standard curve using commercially available recombinant nucleosomes containing histone isoform H3.3 and tested commercially available preparations of recombinant mononucleosomes and polynucleosomes containing histone isoform H3.1. Neither of these preparations gave a significant signal in the H3.3-nucleosome assay. The preparations were diluted from the manufacturer's stated concentration to 1000 ng/ml for testing and the measured results for H3.1-nucleosomes in the H3.3-nucleosome assay were 17.5 ng/ml and 17.3 ng/ml giving cross-reactivities of 1.8% and 1.7% respectively for H3.1-mononucleosomes and H3.1-polynucleosomes. The results are shown in FIG. 2.

As a positive control we tested recombinant nucleosomes containing histone isoform H3.3 supplied from another manufacturer and these H3.3-nucleosomes also gave a significant signal in the H3.3-nucleosome assay (FIG. 2).

We further tested recombinant H3.1-mononucleosomes containing a variety of post-translational modifications (PTMs) including H3.1citrulline, H3.1K27Me3, H3.1K27Ac, H3.1K9Me3, H3.1K4Me2, H3K36Me3 and H3K9Ac for their response in the H3.3-nucleosome assay. We observed a cross-reaction of <2% for all of these nucleosomes except H3.1citrulline which gave a cross-reaction of 8%.

We also tested biologically derived cell free nucleosomes obtained by the digestion of chromatin from cultured HEK293 cells. These cells are rapidly dividing (cells double in culture every 12-48 hours) and would therefore be expected to contain all or predominantly H3.1/2/t-nucleosomes and a low proportion of H3.3-nucleosomes. We observed no significant signal for these nucleosomes in the H3.3-nucleosome assay with a cross reactivity of 1.0% (FIG. 2).

We conclude that the H3.3-nucleosome assay designed to detect and measure intact nucleosomes containing histone H3 isoform H3.3 detects these nucleosomes with little or no detection of nucleosomes containing histone H3 isoforms H3.1, H3.2 and/or H3t.

We also conclude that cell free nucleosomes derived from rapidly dividing HEK293 cells comprise little or no histone H3 isoform H3.3.

### Example 3

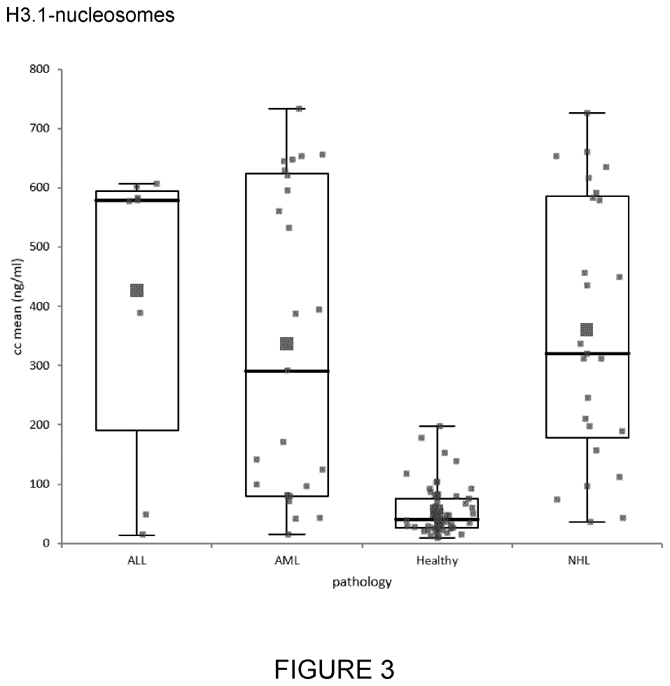

We next tested the invention using EDTA plasma samples obtained from subjects diagnosed with Non-Hodgkin's Lymphoma (NHL). We have previously shown that the level of circulating cell free H3.1-nucleosomes measured in most subjects diagnosed with NHL is highly elevated over the levels measured in healthy subjects (see FIG. 1 in WO2021110776 included here as FIG. 3).

We reasoned that circulating cell free nucleosomes in subjects diagnosed with NHL are likely to be derived predominantly from dead or dying cancer cells and from neutrophils through disease associated inflammation and NETosis. As both cancer cells and neutrophil cells are rapidly dividing, our finding of elevated levels of H3.1-nucleosomes in NHL is consistent with this hypothesis. We further hypothesised that circulating nucleosomes present in NHL are likely to comprise a lower proportion of H3.3-nucleosomes more similar to that found in healthy subjects.

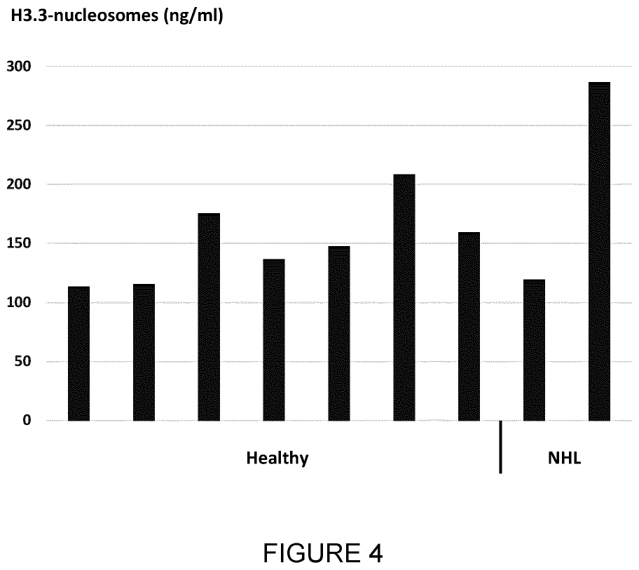

In order to test this hypothesis, we measured the levels of circulating cell free H3.3-nucleosomes present in EDTA plasma samples obtained from 7 healthy subjects and 2 subjects diagnosed with NHL using the H3.3-nucleosome assays described in EXAMPLE 2. The results show that the two NHL samples and the 7 healthy samples contain similar levels of H3.3-nucleosomes (FIG. 4).

### Example 4

We tested whether assays for free histone H3.3 (as opposed to histone H3.3 incorporated as part of a nucleosome) may be used for methods of the invention. We performed Western Blot assays for recombinant H3.3-nucleosomes, recombinant H3.1-nucleosomes, Hela cell nucleosomes and a brain tissue lysate. The histone H3.3 content of each was immunoprecipitated using the capture antibody employed in the prototype double-antibody H3.3-nucleosome assay described in EXAMPLE 2. The isolated histones (free and nucleosome incorporated) were analysed for histone isoform composition by Western blot using a different detection antibody that binds to all histone H3 isoforms.

An analogous Western Blot assay was also performed for histone H3.1 using the capture antibody used in the double-antibody H3.1 assay for immunoprecipitation of histones.

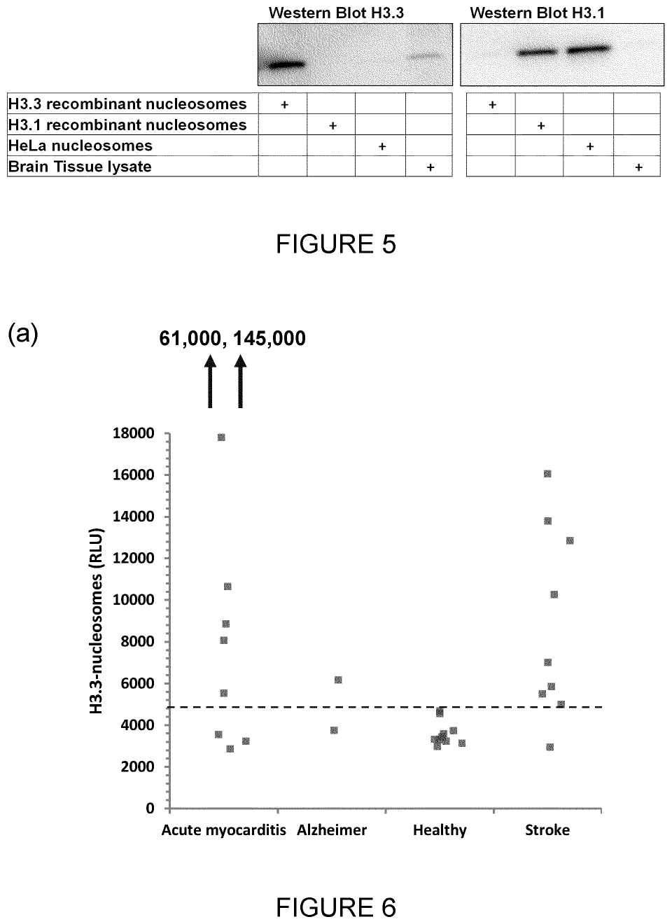

The results are shown in FIG. 5 and show that recombinant H3.3-nucleosomes and the brain tissue lysate tested positive for H3.3 by Western Blot, but recombinant H3.1-nucleosomes and Hela cell nucleosomes (which we deduce to comprise all or mostly histone isoform H3.1 as cancer cells divide rapidly) tested negative.

Similarly, recombinant H3.1-nucleosomes and Hela cell nucleosomes tested positive for H3.1 by Western Blot, but brain tissue lysate and recombinant H3.3-nucleosomes tested negative.

We conclude that both antibodies are specific for binding of their respective histone H3 isoforms. We also conclude that chromatin derived from cancer cells is comprised all or predominantly of histone isoform H3.1, H3.2 or H3t and little or no H3.3. Similarly, the results show that chromatin derived from brain tissue is comprised all or predominantly of histone isoform H3.3 and little or no H3.1.

This Western Blot method measured free histone H3 isoform H3.1 or H3.3 (not nucleosomes) derived from tissue lysate, chromatin or nucleosomes. Therefore, we further conclude that any assay method for histone H3.3, whether in the form of free histone H3 or as part of another structure (for example a chromosome, nucleosome or chromatin fragment) may be used as a method of the invention for the measurement of histone H3.3 in a blood, serum, plasma or other body fluid sample, as a biomarker for the detection or measurement of chromatin with origination in non-dividing cells and/or the death thereof.

### Example 5

We used the prototype sandwich immunoassay described in EXAMPLE 2 to assay H3.3-nucleosome levels in EDTA plasma samples obtained from healthy subjects (n=10) as well as from patients diagnosed with acute myocarditis (n=10), stroke (n=9) and Alzheimer's Disease (n=2). The results are expressed in terms of the output signal of the assay which was Relative Light Units (RLU).

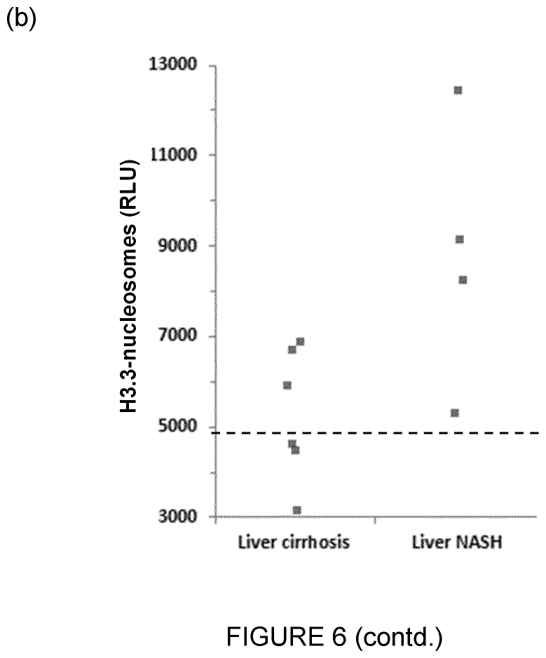

A cut-off representing the upper limit of H3.3-nucleosomes levels in healthy subjects was set at 4800 RLU (higher the level measured in any of the 10 healthy subjects). The results using this cut-off are shown in FIG. 6(a) and show that 7 of 10 patients diagnosed with acute myocarditis, 8 of 9 patients diagnosed with stroke and 1 of 2 patients diagnosed with AD had elevated circulating levels of nucleosomes containing histone isoform H3.3.

In a separate experiment, we also tested patients diagnosed with non-alcoholic steatohepatitis (NASH) (n=4) and liver cirrhosis (n=6). Using the same cut-off we observed that 3 of 6 liver cirrhosis patients and all 4 NASH patients had elevated circulating levels of nucleosomes containing histone isoform H3.3 (FIG. 6(b)).

We conclude that H3.3-nucleosomes originating from tissues comprising slowly dividing cells can be detected in the circulation of patients with diseases of those tissues. We have directly shown this is true for conditions of the central nervous system, liver and heart. Methods of the invention are therefore useful in the detection and monitoring of liver diseases involving the death of liver cells including cirrhosis and NASH, heart disease including acute myocarditis, CNS disorders including stroke, AD and other dementias. Similarly, methods of the invention may be used for the detection of cell death relating to conditions or diseases of the lung and kidney.

### Example 6

We used the prototype sandwich immunoassay described in EXAMPLE 2 to assay H3.3-nucleosome levels in EDTA plasma samples obtained from 6 subjects who suffered a traumatic brain injury. An elevated H3.3-nucleosome level was observed for 4 of the 6 subjects. We conclude that the elevated level of H3.3-nucleosomes circulating in the subjects derived from dead CNS cells caused by injury to the brain. We conclude that methods of the invention may be used to detect and monitor organ injury, including injury to the brain.

### Example 7

Perfusate samples are obtained ex vivo from the perfusion of isolated donor lungs removed for use in transplantation to a recipient. Some lungs are of high quality and are transplanted into a recipient patient. Other lungs are of lower quality and are not used for transplantation. The ex vivo lung perfusate samples are measured for H3.3-nucleosome levels. The H3.3-nucleosome level measured in the perfusates of low-quality lungs is high, reflecting a high level of lung cell death. The H3.3-nucleosome level measured in the perfusates of high-quality lungs is low, reflecting a lower level of lung cell death.

Perfusate samples are obtained ex vivo from the perfusion of isolated donor livers removed for use in transplantation to a recipient. Some livers are of high quality and are transplanted into a recipient patient. Other livers are of lower quality and are not used for transplantation. The ex vivo liver perfusate samples are measured for H3.3-nucleosome levels. The H3.3-nucleosome level measured in the perfusates of low-quality liver perfusates is high, reflecting a high level of liver cell death. The H3.3-nucleosome level measured in the perfusates of high-quality livers is low, reflecting a lower level of liver cell death.

Therefore methods of the invention can be used to monitor the condition of donor organs prior to transplantation and to ascertain their quality and suitability for transplantation into a recipient patient.

### Example 8

Serial plasma samples are obtained from lung, liver and kidney transplant recipients at time intervals following transplantation. Recipient circulating H3.3-nucleosome levels are observed to increase prior to rejection or other conditions associated with the transplanted organ. Recipient circulating H3.3-nucleosome levels can therefore be used as a biomarker for the health or deterioration of the transplanted organ in the recipient.

### Example 9

Experiments similar to those described in EXAMPLES 5-8 are performed in which free histone H3.3 is measured by Western Blot in place of a 2-antibody immunoassay for nucleosomes containing histone H3.3. The Western Blot results obtained are substantially similar to the immunoassay results, indicating that histone isoform H3.3 may be used as a biomarker for death of non-dividing or slowly dividing cells in conditions in which such cell death occurs and that histone isoform H3.3 may be measured in any form (including for example as part of a nucleosome or as a single histone moiety) in methods of the invention.

### REFERENCES

Ahmad and Henikoff. PNAS. 2002 vol. 99 16477-16484. doi 10.1073/pnas. 172403699
Ausio. Biochem Cell Bio 2001. 79:693
Barefoot et al. Front. Genet. 2021. 12:671057. doi: 10.3389/fgene.2021.671057
Brent and Schultz, Proc. Natl. Acad. Sci. USA. 1999. Vol. 96, pp. 1345-1350, Biochemistry
Commerford et al. Proc. Natl Acad. Sci. USA. 1982. Vol. 79, pp. 1163-1165, Cell Biology
Cavalier et al. Front. Mol. Biosci. 8:600881. doi: 103389/fmob.2021.600881.
Dengu et al. Annual Congress 2022 Int Liver Transplant Society Istanbul May 4-7 2022
Guseh. Hum Genet 2020 September; 139(9):1141-1148. doi: 10.1007/s00439-019-02061-1.
Herranz and Esteller, Methods Mol. Biol. (2007) 361:25-62
Holdenrieder et al. Int. J. Cancer (Pred. Oncol.): 95, 114-120 (2001)
Liu et al. Br J Surg. 2017. 104(9):1215-1225. doi: 10.1002/bjs. 10538
Maze et al. Neuron. 2015. 87, 77-94, doi.org/10.1016/j.neuron.2015.06.014
Morimont et al. Biomolecules 2022, 12, 1038. doi.org/10.3390/biom12081038
Pantel K (2016) PLoS Med 13(12): e1002205
Pietronigro et al. Front. Immunol. 2017. doi: 10.3389/fimmu.2017.00211
Polivka et al. Expert Rev Mol Diagn. 2015. doi: 10. 1586/14737159.2015.1110021
Seim et al. npj Aging Mechanisms Disease (2016) 2, 16014; doi: 10.1038/npjamd.2016.14
Snyder et al. Cell. 2016; 164(1-2):57-68. doi: 10.1016/j.cell.2015.11.050
Thongprayoon et al. J Clin Med. 2020 May; 9(5): 1480. doi: 10.3390/jcm9051480
Tvardovskiy et al. Nucleic Acids Res, 2017, Vol. 45, 9272-9289, doi: 10. 1093/nar/gkx696
Van den Ackerveken et al. Sci Rep. 2021. 11, 7256 doi.org/10.1038/s41598-021-86630-3.
Wang et al. Front. Cell. Neurosci. 13:199. doi: 10.3389/fncel.2019.00199
Yi and Kim, BMB Rep (2018) 51:211-218. doi: 10.5483/bmbrep.2018.51.5.053.
Zhao and Garcia (2015) Cold Spring Harb Perspect Biol, 7: a025064

1-3. (canceled)

4. A method of detecting whether a cell free histone or cell free nucleosome is derived from a dividing cell or a non-dividing cell, comprising:
contacting a body fluid sample obtained from an individual with a first binding agent which specifically binds to a H3.1, H3.2 or H3t histone protein and a second binding agent which specifically binds to a H3.3 histone protein, and
detecting whether a cell free histone or cell free nucleosome is derived from a non-dividing cell or a dividing cell based on the presence or level of such binding to the H3.1, H3.2 or H3t histone protein and/or to the H3.3 histone protein, wherein:
(i) the presence of binding to the H3.3 histone protein or an increased level of binding to the H3.3 histone protein relative to the level of binding to the H3.1, H3.2 or H3t histone protein is indicative that the cell free histone or cell free nucleosome is derived from a non-dividing cell; and
(ii) the presence of binding to the H3.1, H3.2 or H3t histone protein or an increased level of binding to the H3.1, H3.2 or H3t histone protein relative to the level of binding to the H3.3 histone protein is indicative that the cell free histone or cell free nucleosome is derived from a dividing cell.

5. The method of claim 4, wherein the non-dividing cell is selected from the group consisting of (i) a stable cell or a permanent cell, (ii) a cell which has a cell turnover of greater than or equal to 200 days, (iii) a brain cell, (iv) a heart muscle cell, (v) a kidney cell, (vi) a liver cell, (vii) a lung cell, (viii) a cell capable of mitosis, (ix) a labile cell, (x) a white blood cell.

6-11. (canceled)

12. A method of assessing a disease condition in an individual, comprising:
contacting a body fluid sample obtained from the individual with a first binding agent which specifically binds to a H3.1, H3.2 or H3t histone protein and a second binding agent which specifically binds to a H3.3 histone protein, and
assessing the disease condition based on the presence or level of such binding to the H3.1, H3.2 or H3t histone protein and/or the H3.3 histone protein, wherein:
(i) the presence of binding to the H3.1, H3.2 or H3t histone protein or an increased level of binding to the H3.1, H3.2 or H3t histone protein relative to the level of binding to the H3.3 binding protein is indicative that the disease condition is associated with inflammation;
(ii) the presence of binding to the H3.1, H3.2 or H3t histone protein or an increased level of binding to the H3.1, H3.2 or H3t histone protein relative to the level of binding to the H3.3 binding protein is indicative that the disease condition is associated with neutrophil extracellular traps (NETs) or extracellular traps (ETs); and/or
(iii) the presence of binding to the H3.3 histone protein or an increased level of binding to the H3.3 histone protein relative to the level of binding to the H3.1, H3.2 or H3t histone protein is indicative that the disease condition is characterised by death of non-dividing cells.

13. The method of claim 12, wherein the disease condition is an infection or a reaction to an infection.

14. The method of claim 13, wherein the reaction to an infection is sepsis.

15. The method of claim 12, wherein the disease condition is COVID-19.

16. The method of claim 12, wherein the disease condition is associated with organ failure.

17-23. (canceled)

24. The method of claim 12, wherein the body fluid sample is selected from cerebrospinal fluid, blood, serum and plasma sample.

25-28. (canceled)

29. A method for isolating nucleosomes and/or nucleic acid from a tumour, comprising:
(i) contacting a body fluid sample obtained from a subject with a binding agent which specifically binds to a H3.3 histone protein characterised by a nucleosome;
(ii) isolating the nucleosomes not bound to the binding agent; and
(iii) optionally extracting the nucleic acid characterised by the nucleosomes isolated in step (ii).

30. The method of claim 29, wherein the tumour is not a tumour of the CNS.

31-42. (canceled)

43. The method of claim 29, wherein the body fluid sample is selected from the group consisting of cerebral spinal fluid, blood, serum and plasma.

44. (canceled)

45. The method of claim 29, wherein the H3.1, H3.2 or H3t histone protein and/or the H3.3 histone protein is characterised by a nucleosome, an NET or an ET.

46. The method of claim 29, wherein any first and second binding agents are added to the sample separately in any order or are added simultaneously.

47. The method of claim 29, wherein the method is repeated on one or more occasions and any changes in the level of binding to the first binding agent and/or the second binding agent is used to monitor the progression of the disease condition in the individual.

48. The method of claim 29, wherein the method is performed using chromatin immunoprecipitation.

49. The method of claim 29, wherein the histone protein is post-translationally modified.

50-53. (canceled)
